(19) United States
(12) Patent Application Publication
(10) Pub. No.: US 2017/0151264 A1
(43) Pub. Date: Jun. 1, 2017

(54) COMBINATION

(71) Applicant: Almirall, S.A., Barcelona (ES)

(72) Inventors: Nuria GODESSART MARINA, Barcelona (ES); Cristina BALAGUE PELAEZ, Barcelona (ES)

(21) Appl. No.: 15/313,737

(22) PCT Filed: May 21, 2015
(86) PCT No.: PCT/EP2015/061312

§ 371 Date: Nov. 23, 2016

(30) Foreign Application Priority Data
May 27, 2014 (EP) 14382192.4
Oct. 17, 2014 (EP) 14382400.1
Oct. 17, 2014 (EP) 14382401.9

(51) Int. Cl. A61K 31/573; A61K 45/06; A61K 31/53

(52) U.S. Cl. CPC A61K 31/573; A61K 31/53; A61K 45/06

(57) ABSTRACT

The present invention provides a pharmaceutical composition which comprises (a) a compound which is an inhibitor of phosphoinositide 3-kinase delta or a pharmaceutically acceptable salt and/or solvate thereof, and (b) a corticosteroid.

### FIELD OF THE INVENTION

The present invention is directed to new combination therapies involving phosphoinositide 3-Kinase delta (PI3K delta) inhibitors and corticosteroids. Such combinations are useful in the treatment of skin diseases, in particular immunobullous skin diseases mediated by autoantibodies, and specifically pemphigus vulgaris.

### BACKGROUND OF THE INVENTION

Immunobullous skin diseases mediated by autoantibodies (also known as autoimmune blistering diseases or AIBDs) are a group of rare skin disorders characterized by IgG (or less often IgA) autoantibodies that attack adhesive proteins of the epidermis or the dermal-epidermal junction. These disorders present as blisters and erosions of the skin and/or mucous membranes. They can affect individuals of any age including children. In Germany, there are an estimated 2000 new cases of AIBDs per year, with an overall prevalence of about 12,000 cases. The incidence of the related diseases epidermolysis bullosa acquista (EBA) and the pemphigoid disorders is around 1 and 25 new cases per/million residents, respectively (Schmidt E, Zillikens D. Dermatol Clin 2011; 29:663-71; Joly P. J Inv Derm 2012; 132: 1998-04; Bertram F. J. Dtsch Derm Ges 2009; 7: 434-9.).

Immunobullous skin diseases mediated by autoantibodies are well known in the art and include intraepidermal immunobullous diseases, such as pemphigus vulgaris, pemphigus vegetans, pemphigus foliaceus, endemic pemphigus foliaceus, intercellular IgA dermatosis, paraneoplastic pemphigus; and subepidermal immunobullous diseases, such as bullous pemphigoid, mucous membrane pemphigoid, pemphigoid gestationis, linear IgA disease, epidermolysis bullosa acquisita, bullous systemic lupus erythematosus and dermatitis herpetiformis.

Pemphigus is a chronic immunobullous skin disease mediated by autoantibodies that causes painful blisters on skin and mucosae. The two main types of pemphigus are p. vulgaris (PV) and p. foliaceus and both are potentially lethal. PV is the most common form of pemphigus in the EU, accounting for 70-80% of all cases (Schmidt E, Zillikens D. Dermatol Clin 2011; 29:663-71; Joly P. J Inv Derm 2012; 132: 1998-04; Bertram F. J. Dtsch Derm Ges 2009; 7: 434-9.). Patients develop blisters that break almost immediately, leaving ulcerated sores. Both, cutaneous and mucosal lesions, are slow to heal, lead to major general discomfort, loss of body proteins, increase susceptibility to infection, and difficulties in eating and drinking (Kneisel A, Hertl M. J. Dtsch Derm Ges. 2011; 9(10):844-56).

Most pemphigus forms display serum IgG autoantibodies that target desmogleins (Dsg), which are components of desmosomes (adhesive complexes between keratinocytes) and induce loss of cell adhesion, eventually leading to blistering. Autoantibody-induced impairment of distinct Dsg isoforms causes either the mucosal form of PV (anti-Dsg3 IgG, oral mucosa lesions only), the mucocutaneous form of PV (anti-Dsg3 and anti-Dsg1 IgG, oral and skin lesions) or p. foliaceus (anti-Dsg1 IgG, skin lesions only). PV can be regarded as a prototypical B cell-mediated autoimmune disease where pathogenic IgG autoantibodies are the direct cause of the symptoms (Kneisel A, Hertl M. J. Dtsch Derm Ges. 2011; 9(11):927-47; Joly P. Clin Dermatol. 2011; 29(4):432-6.).

Pemphigus is estimated to affect anywhere from 0.7 to 5 people per 1,000,000 per year in the general population (NORD Rare Diseases Data Base, accessed October 2014). The incidence and proportion varies between territories (Meyer N, Misery L. Autoimmunity Reviews 2010; 9: A379-A382), but it is more prevalent in people of the Mediterranean area or Jewish ancestry. Men and women are equally affected. Although the onset usually occurs in middle-aged adults, the disease may also appear in young adults and children.

There is currently no cure for pemphigus vulgaris. The primary aim of current treatment is to decrease blister formation, prevent infections and promote healing of blisters and erosions. High dose corticosteroids (CS) are the standard of care (SOC) treatment for PV. CS act quickly and provide symptom relief, with long-term use being required to prevent relapses (maintenance of remission). However, 50% of patients remain poorly controlled after 1 year of treatment (Herbst A, Bystryn J C. J Am Acad Dermatol 2000; 42 (3), 422-427). In addition, long-term use of high dose CS increases the risks of side effects (morbidities and risk of mortality). To palliate this, adjuvant therapies are used as CS-sparing drugs to reduce CS side effects (azathioprine, mycophenolate mofetil, rituximab, methotrexate, IgG, cyclophosphamide, cyclosporine) but have not provided any additional efficacy over CS alone. Currently, there is a lack of alternative treatment to PV, with an improved efficacy/balance over current SOC.

The mortality rate of PV is about 5-15% (Schmidt E, Zillikens D. Dermatol Clin 2011; 29:663-71; Joly P. J Inv Derm 2012; 132: 1998-04; Bertram F. J. Dtsch Derm Ges 2009; 7: 434-9.). Mortality in patients with PV is 3 times higher than the general population, mainly due to the side effects of the current standard of care (SOC), high-dose CS including peptic ulcer disease and GI bleeds, and susceptibility to infection with sepsis. Morbidity and mortality are related to the extent of disease, the maximum dose of CS required to induce remission, and the presence of other diseases. Current morbidity of PV is largely iatrogenic, caused by side effects of the long-term high-dose CS and immunosuppressive adjuvants.

New and more effective therapies are therefore needed in the treatment of immunobullous skin diseases mediated by autoantibodies, in particular pemphigus vulgaris.

Phosphoinositide 3-Kinase delta (PI3K delta) inhibitors are effective in the treatment of immunobullous skin diseases mediated by autoantibodies, in particular pemphigus vulgaris. Further, it is a finding of the present invention that PI3K delta inhibitors are more effective when administered together with a corticosteroid. Specifically, the PI3K delta inhibitor and the corticosteroid achieve a greater than additive effect in achieving efficacy against immunobullous skin diseases mediated by autoantibodies, in particular pemphigus vulgaris. Thus, the efficacy achieved with the drug combination of the present invention is greater than that which would be expected from consideration of the activity of each monotherapy.

This new and surprising finding will enable new treatment options for immunobullous skin diseases mediated by autoantibodies which will allow for a reduction in the dose of corticosteroids needed (with consequent reduction of side-effects) and will avoid the need for adjuvant immunosuppressant/immunomodulatory drugs, which are also associated with detrimental side-effects.

### SUMMARY OF THE INVENTION

The present invention therefore provides a pharmaceutical composition which comprises (a) a compound which is an inhibitor of phosphoinositide 3-kinase delta or a pharmaceutically acceptable salt and/or solvate thereof, and (b) a corticosteroid.

Also provided is a composition of the invention for use in the treatment of the human or animal body.

Also provided is a composition of the invention for use in treating a skin disease as defined herein, typically an immunobullous skin disease mediated by autoantibodies as defined herein.

Also provided is use of a composition of the invention in the manufacture of a medicament for use in the treatment of the human or animal body.

Also provided is use of a composition of the invention in the manufacture of a medicament for use in treating a skin disease as defined herein, typically an immunobullous skin disease mediated by autoantibodies as defined herein.

Also provided is a product comprising (a) a compound which is an inhibitor of phosphoinositide 3-kinase delta or a pharmaceutically acceptable salt and/or solvate thereof as defined herein and (b) a corticosteroid as defined herein, optionally together with at least one other active compound as defined herein, for simultaneous, concurrent, separate or sequential use in the treatment of the human or animal body.

Also provided is a product comprising (a) a compound which is an inhibitor of phosphoinositide 3-kinase delta or a pharmaceutically acceptable salt and/or solvate thereof as defined herein and (b) a corticosteroid as defined herein, optionally together with at least one other active compound as defined herein, for simultaneous, concurrent, separate or sequential use in the treatment of a skin disease as defined herein.

Also provided is a combination comprising (a) a compound which is an inhibitor of phosphoinositide 3-kinase delta or a pharmaceutically acceptable salt and/or solvate thereof as defined herein and (b) a corticosteroid as defined herein, optionally together with at least one other active compound as defined herein, for simultaneous, concurrent, separate or sequential use in the treatment of the human or animal body.

Also provided is a combination comprising (a) a compound which is an inhibitor of phosphoinositide 3-kinase delta or a pharmaceutically acceptable salt and/or solvate thereof as defined herein and (b) a corticosteroid as defined herein, optionally together with at least one other active compound as defined herein, for simultaneous, concurrent, separate or sequential use in the treatment of a skin disease as defined herein.

Also provided is use of (a) a compound which is an inhibitor of phosphoinositide 3-kinase delta or a pharmaceutically acceptable salt and/or solvate thereof as defined herein and (b) a corticosteroid as defined herein in the preparation of a medicament, for simultaneous, concurrent, separate or sequential use in the treatment of the human or animal body.

Also provided is use of (a) a compound which is an inhibitor of phosphoinositide 3-kinase delta or a pharmaceutically acceptable salt and/or solvate thereof as defined herein and (b) a corticosteroid as defined herein in the preparation of a medicament, for simultaneous, concurrent, separate or sequential use in the treatment of a skin disease as defined herein.

Also provided is a compound which is an inhibitor of phosphoinositide 3-kinase delta or a pharmaceutically acceptable salt and/or solvate thereof as defined herein for use in the treatment of the human or animal body, by simultaneous, concurrent, separate or sequential use in combination with a corticosteroid as defined herein and optionally at least one other active compound, as defined herein. Typically, treatment of the human or animal body is treatment of a skin disease as defined herein.

Also provided is a corticosteroid as defined herein for use in the treatment of the human or animal body, by simultaneous, concurrent, separate or sequential use in combination with a compound which is an inhibitor of phosphoinositide 3-kinase delta or a pharmaceutically acceptable salt and/or solvate thereof as defined herein and optionally at least one other active compound, as defined herein. Typically, treatment of the human or animal body is treatment of a skin disease as defined herein.

Also provided is use of a compound which is an inhibitor of phosphoinositide 3-kinase delta or a pharmaceutically acceptable salt and/or solvate thereof as defined herein in the manufacture of a medicament for use in the treatment of the human or animal body by simultaneous, concurrent, separate or sequential use in combination with a corticosteroid as defined herein and optionally at least one other active compound, as defined herein. Typically, treatment of the human or animal body is treatment of a skin disease as defined herein.

Also provided is use of a corticosteroid as defined herein in the manufacture of a medicament for use in the treatment of the human or animal body by simultaneous, concurrent, separate or sequential use in combination with a compound which is an inhibitor of phosphoinositide 3-kinase delta or a pharmaceutically acceptable salt and/or solvate thereof as defined herein and optionally at least one other active compound, as defined herein. Typically, treatment of the human or animal body is treatment of a skin disease as defined herein.

Also provided is a method of treatment of a patient in need thereof, which method comprises administering to said patient a combination or composition as defined herein.

Also provided is a method of treatment of a patient suffering from a skin disease as defined herein, which method comprises administering to said patient a combination or composition as defined herein.

Also provided is a kit of parts comprising a compound which is an inhibitor of phosphoinositide 3-kinase delta or a pharmaceutically acceptable salt and/or solvate thereof as defined herein, together with instructions for simultaneous, concurrent, separate or sequential use in combination with a corticosteroid as defined herein and optionally at least one other active compound as defined herein for the treatment of a human or animal patient.

Also provided is a kit of parts comprising a compound which is an inhibitor of phosphoinositide 3-kinase delta or a pharmaceutically acceptable salt and/or solvate thereof as defined herein, together with instructions for simultaneous, concurrent, separate or sequential use in combination with a corticosteroid as defined herein and optionally at least one other active compound as defined herein for the treatment of a human or animal patient suffering from or susceptible to a skin disease as defined herein.

Also provided is a package comprising a compound which is an inhibitor of phosphoinositide 3-kinase delta or a pharmaceutically acceptable salt and/or solvate thereof as defined herein, and a corticosteroid as defined herein and optionally at least one other active compound as defined herein for the simultaneous, concurrent, separate or sequential use in the treatment of a human or animal patient.

Also provided is a package comprising a compound which is an inhibitor of phosphoinositide 3-kinase delta or a pharmaceutically acceptable salt and/or solvate thereof as defined herein, and a corticosteroid as defined herein and optionally at least one other active compound as defined herein for the simultaneous, concurrent, separate or sequential use in the treatment of a human or animal patient suffering from or susceptible to a skin disease as defined herein.

Also provided is use of (a) a compound which is an inhibitor of phosphoinositide 3-kinase delta or a pharmaceutically acceptable salt and/or solvate thereof as defined herein for the preparation of a medicament, for simultaneous, concurrent, separate or sequential use in combination with (b) a corticosteroid as defined herein for the treatment of the human or animal body.

Also provided is use of (a) a compound which is an inhibitor of phosphoinositide 3-kinase delta or a pharmaceutically acceptable salt and/or solvate thereof as defined herein for the preparation of a medicament, for simultaneous, concurrent, separate or sequential use in combination with (b) a corticosteroid as defined herein for the treatment of a skin disease as defined herein, preferably an immunobullous skin disease mediated by autoantibodies as defined herein.

Also provided is use of (b) a corticosteroid as defined herein for the preparation of a medicament, for simultaneous, concurrent, separate or sequential use in combination with (a) a compound which is an inhibitor of phosphoinositide 3-kinase delta or a pharmaceutically acceptable salt and/or solvate thereof as defined herein for the treatment of the human or animal body.

Also provided is use of (b) a corticosteroid as defined herein for the preparation of a medicament, for simultaneous, concurrent, separate or sequential use in combination with (a) a compound which is an inhibitor of phosphoinositide 3-kinase delta or a pharmaceutically acceptable salt and/or solvate thereof as defined herein for the treatment of a skin disease as defined herein, preferably an immunobullous skin disease mediated by autoantibodies as defined herein.

Also provided is a method of treatment of a patient in need thereof, which method comprises administering a combination or composition as defined herein.

Also provided is a method of treatment of a patient suffering from or susceptible to a skin disease as defined herein, preferably an immunobullous skin disease mediated by autoantibodies as defined herein, which method comprises administering a combination or composition as defined herein.

### BRIEF DESCRIPTION OF THE FIGURES

FIG. 1 shows the effect of a representative compound of the invention, LAS191954, and prednisolone on the kinetics of antibody production to Dsg3.

FIG. 2 shows the effect of a representative compound of the invention, LAS191954, and prednisolone on the kinetics of antibody production to dsDNA.

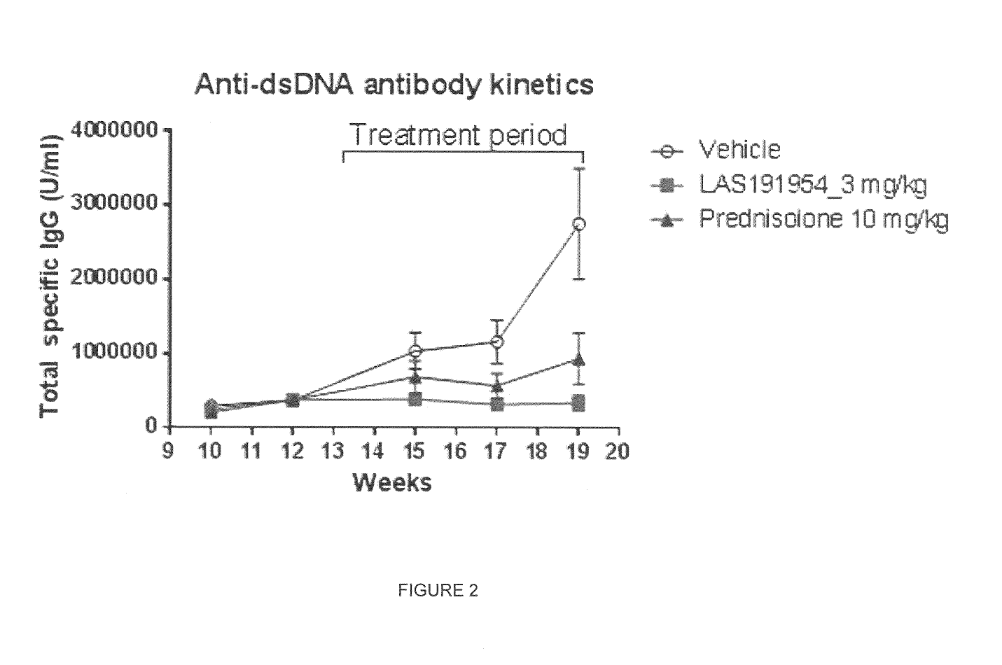

FIG. 3 shows the relative change of Anti-Dsg3 (Left) and Anti-dsDNA (Right) antibody levels in a spontaneous autoimmune disease model.

FIG. 4 shows the effect of a representative compound of the invention, LAS191954, on clinical disease in established experimental EBA as determined by the percentage of body surface area affected by skin lesions in relation to the score at inclusion to treatment.

FIG. 5 shows the effect of a representative compound of the invention, LAS191954, on clinical disease in established experimental EBA as determined by the overall disease activity, expressed as AUC derived from graphs in FIG. 4.

FIG. 6 shows the effect of a representative compound of the invention, LAS191954, on clinical disease in established experimental EBA as determined by representative clinical manifestations.

FIG. 7 shows the effect of a representative compound of the invention, LAS191954, on body weight gain in established experimental EBA.

FIG. 8 shows the effect of combining different doses of LAS191954 and prednisolone on the kinetics of autoantibody production to Dsg3 in a murine model of autoimmune disease. Statistical significance being calculated with 2-Way ANOVA using Tukey's post-test analysis (*p<0.05; **p<0.01; ***p<0.001)

### DETAILED DESCRIPTION OF THE INVENTION

The term “therapeutically effective amount” refers to an amount sufficient to effect treatment when administered to a patient in need of treatment.

The term “treatment” as used herein refers to the treatment of a disease or medical condition in a human or animal patient which includes:

(a) preventing the disease or medical condition from occurring, i.e., prophylactic treatment of a patient;
(b) ameliorating the disease or medical condition, i.e., causing regression of the disease or medical condition in a patient;
(c) suppressing the disease or medical condition, i.e., slowing the development of the disease or medical condition in a patient; and/or
(d) alleviating the symptoms of the disease or medical condition in a patient.

The term “pharmaceutically acceptable salt” refers to a salt prepared from a base or acid which is acceptable for administration to a patient, such as a human or animal e.g. a mammal. Such salts can be derived from pharmaceutically-acceptable inorganic or organic bases and from pharmaceutically-acceptable inorganic or organic acids.

Pharmaceutically acceptable acids include both inorganic acids, for example hydrochloric, sulphuric, phosphoric, diphosphoric, hydrobromic, hydroiodic and nitric acid; and organic acids, for example citric, fumaric, gluconic, glutamic, lactic, maleic, malic, mandelic, mucic, ascorbic, oxalic, pantothenic, succinic, tartaric, benzoic, acetic, methanesulphonic acid, ethanesulphonic, benzenesulphonic, naphthalene-2-sulfonic acid, p-toluenesulphonic acid, xinafoic (1-hydroxy-2-naphthoic acid), napadisilic (1,5-naphthalenedisulfonic acid) and the like. Particularly preferred are salts derived from methanesulphonic acid, naphthalene-2-sulfonic acid, and p-toluenesulphonic acid.

Salts derived from pharmaceutically-acceptable inorganic bases include aluminum, ammonium, calcium, copper, ferric, ferrous, lithium, magnesium, manganic, manganous, potassium, sodium, zinc and the like.

Salts derived from pharmaceutically-acceptable organic bases include salts of primary, secondary and tertiary amines, including alkyl amines, arylalkyl amines, heterocyclyl amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

The term “solvate” refers to a complex or aggregate formed by one or more molecules of a solute, i.e. a phosphoinositide 3-kinase delta inhibitor or a pharmaceutically acceptable salt thereof, and one or more molecules of a solvent. Such solvates are typically crystalline solids having a substantially fixed molar ratio of solute and solvent. Representative solvents include by way of example, water, acetone, dichloromethane, 2-propanol, ethanol, methanol, dimethylsulfoxide (DMSO), ethyl acetate, acetic acid, ethanolamine, or mixtures thereof. When the solvent is water, the solvate formed is a hydrate. It is specifically contemplated that one solvent molecule can be associated with one molecule of a phosphoinositide 3-kinase delta inhibitor or a pharmaceutically acceptable salt thereof, such as a hydrate. Furthermore, it is specifically contemplated that more than one solvent molecule may be associated with one molecule of a phosphoinositide 3-kinase delta inhibitor or a pharmaceutically acceptable salt thereof, such as a dihydrate. Additionally, it is specifically contemplated that less than one solvent molecule may be associated with a phosphoinositide 3-kinase delta inhibitor or a pharmaceutically acceptable salt thereof, such as a hemihydrate. Furthermore, solvates are contemplated as solvates of a phosphoinositide 3-kinase delta inhibitor or a pharmaceutically acceptable salt thereof that retain the biological effectiveness of the non-solvate form of the compounds.

The term “pharmaceutically (or physiologically) acceptable carrier (or diluent)” refers to a carrier or diluent that does not cause significant irritation to an organism and does not abrogate the biological activity and properties of the administered compound.

As used herein, the term “an inhibitor of phosphoinositide 3-kinase delta” refers to a compound that demonstrates activity against expression of phosphoinositide 3-kinase delta in a suitably chosen assay method, for instance an assay based on M-CSF-induced AKT phosphorylation, a downstream effector of PI3Kδ, in THP-1 cells.

Typically, “an inhibitor of phosphoinositide 3-kinase delta” refers to a compound that possesses an IC₅₀ value for the inhibition of PI3Kδ of less than 10 μm, preferably less than 1 μm, even more preferably less than 0.2 μm, most preferably less than 0.05 μm, for instance in the assay method referred to above.

Typically, the term “an inhibitor of phosphoinositide 3-kinase delta” refers to a compound that inhibits the activity of the PI3K delta isozyme more effectively than other isozymes of the PI3K family (alpha, beta, and gamma). For instance, the PI3-kinase delta selective inhibitor may refer to a compound that exhibits a 50 percent inhibitory concentration (IC₅₀) with respect to the delta type PI3-kinase that is at least 10-fold, preferably at least 20-fold, more preferably at least 50-fold, most preferably at least 100-fold or lower than the inhibitor's IC50 with respect to the rest of the other type PI3-kinases (i.e., alpha, beta, and gamma). Typically, this selectivity is determined using an assay method as defined above.

Typically, the inhibitor of phosphoinositide 3-kinase delta is a compound as defined in WO-A-2012/146666, the entirety of which is incorporated herein by reference.

Thus, typically, the inhibitor of phosphoinositide 3-kinase delta is of formula (I)

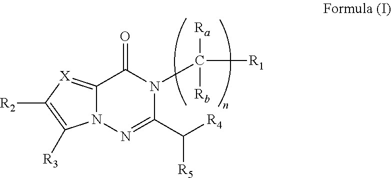

wherein X, Rₐ, R_{b}, n, R₁, R₂, R₃, R₄ and R₅ are as defined in WO-A-2012/146666.

Preferably, in the compound of formula (I):
X represents a nitrogen atom or a —CR₆ group;
Rₐ and R_{b} each independently represent a hydrogen atom or a methyl group;
R₁ represents a hydrogen atom, a halogen atom, a C₁-C₃ haloalkyl group, a methyl group, a C₃-C₇ cycloalkyl group, a phenyl group, a pyridinyl group, a pyrazolyl group, an isoxazolyl group, a piperidinyl group or a tetrahydropyranyl group; wherein the cycloalkyl, phenyl, pyridinyl, pyrazolyl, isoxazolyl, piperidinyl or tetrahydropyranyl groups are unsubstituted or substituted by one or more substituents selected from a halogen atom, a hydroxyl group, a C₁-C₃ haloalkyl group, a linear or branched C₁-C₃ alkyl group, a —(CH₂)-(phenyl)-O—(C₁-C₃ alkyl group), a —NR₇R₈ group or a —OR₈ group; wherein R₇ and R₈ each independently represent a hydrogen atom or a linear or branched C₁-C₃ alkyl group;
R₂ and R₃ each independently represent a hydrogen atom, a halogen atom, a cyano group, a C₁-C₃ haloalkyl group or a linear or branched C₁-C₃ alkyl group;
R₄ represents a hydrogen atom, a C₁-C₃ haloalkyl group, a C₁-C₃ hydroxyalkyl group or a linear or branched C₁-C₃ alkyl group;
R₆ represents a hydrogen atom, a halogen atom, a C₁-C₃ haloalkyl group, a linear or branched C₁-C₃ hydroxyalkyl group, a linear or branched C₁-C₃ alkyl group or a cyclopropyl group;
R₆ represents a hydrogen atom; a halogen atom; a hydroxyl group; a cyano group; a C₁-C₄ alkoxy group; a C₁-C₄ haloalkyl group; a linear or branched C₁-C₄ hydroxyalkyl group; a C₃-C₇ cycloalkyl group; a linear or branched C₁-C₃ alkyl group;
a —(CH₂)₀₋₃NR′R″ group; a —(CH₂)₁₋₃O(C₁-C₃ alkyl group); a —(CH₂)₀₋₃OC(CH₂)—(C₁-C₃ alkyl group); a —(CH₂)₀₋₃C(O)O—(C₁-C₃ alkyl group); a —C(O)—NR′R″ group; a —(CH₂)₀₋₃C(O)OH group; a —(CH₂)₀₋₃-(imidazolyl group); a —(CH₂)₀₋₃-(oxazolyl group); a —(CH₂)₀₋₃-(oxadiazolyl group); a —(CH₂)₀₋₃-(pyrazolyl group) or a —(CH₂)₀₋₃-(morpholinyl group); wherein R′ and R″ each independently represent a hydrogen atom, a hydroxyl group, or a linear or branched C₁-C₃ alkyl group; and
wherein the imidazolyl, oxazolyl, oxadiazolyl, pyrazolyl and morpholinyl groups are unsubstituted or substituted by one or more substituents selected from a halogen atom, a linear or branched C₁-C₃ alkyl group or a C₁-C₃ haloalkyl group.
R₅ represents a group selected from:
i) a group of formula (IIa), which group is a purinyl group unsubstituted or substituted by a —NR′R″ group;
ii) a group of formula (IIb), which group is selected from a —NR′-pyridinyl group,
a —S-pyridinyl group, a —NR′-pyrimidinyl group, a —S-pyrimidinyl group or
a —NR′-triazinyl group; wherein the pyridinyl, pyrimidinyl and triazinyl groups are unsubstituted or substituted by one, two or three substituents selected from a halogen atom, a C₁-C₃ haloalkyl group, a —(CH₂)₀₋₃CN group, a —C(O)—(CH₂)₀₋₃—NR′R″, a —(CH₂)₀₋₃NR′R″ group; and
iii) a group of formula (IIc), which group is selected from a —NR′-purinyl group, a —S— purinyl group, a —NR′-7H-pyrrolo[2,3-d]pyrimidinyl group, a —NR′-1H-pyrazolo[3,4-d]pyrimidinyl group or a —NR′-pyrazolo[1,5-a]pyrimidinyl group; wherein the purinyl, 7H-pyrrolo[2,3-d]pyrimidinyl, 1H-pyrazolo[3,4-d]pyrimidinyl pyrazolo[1,5-a]pyrimidinyl and groups are unsubstituted or substituted by a halogen atom or a —(CH₂)₀₋₃NR′R″ group; or
R₄ and R₅ together with the carbon atom to which they are attached form a pyrrolidinyl-purinyl group or a pyrrolidinyl-pyrimidinyl; wherein the pyrrolidinyl group is unsubstituted or substituted by one or more substituents selected from a halogen atom or a hydroxyl group; and wherein the purinyl group is unsubstituted or substituted by
a —(CH₂)₀₋₃NR′R″ group; and wherein the pyrimidinyl group is unsubstituted or substituted by one, two or three substituents selected from a —(CH₂)₀₋₃CN group or a —(CH₂)₀₋₃NR′R″ group; and
R′ and R″ each independently represent a hydrogen atom, a C₁-C₃ alkoxy group or a linear or branched C₁-C₃ alkyl group.

Alternatively in the compound of formula (I):
X represents a nitrogen atom or a —CR₆ group;
Rₐ and R_{b} each independently represent a hydrogen atom or a methyl group;
R₁ represents a methyl group, a C₃-C₇ cycloalkyl group, a phenyl group, a pyridinyl group, a piperidinyl group or a tetrahydropyranyl group;
wherein the cycloalkyl, phenyl, pyridinyl, piperidinyl or tetrahydropyranyl groups are unsubstituted or substituted by one or more substituents selected from a halogen atom, a linear or branched C₁-C₃ alkyl group, a —NR₇R₈ group or a —OR₈ group; wherein R₇ and R₈ each independently represent a hydrogen atom or a linear or branched C₁-C₃ alkyl group;
R₂ and R₃ each independently represent a hydrogen atom or a linear or branched C₁-C₃ alkyl group;
R₄ represents a hydrogen atom, a C₁-C₃ haloalkyl group, or a linear or branched C₁-C₃ alkyl group;
R₆ represents a hydrogen atom, a halogen atom, a C₁-C₃ haloalkyl group, a linear or branched C₁-C₃ alkyl group or a cyclopropyl group;
R₅ represents a group selected from:
i) a group of formula (IIa), which group is a purinyl group unsubstituted or substituted by a —NR′R″ group;
ii) a group of formula (IIb), which group is selected from a —NH-pyridinyl group,
a —S-pyridinyl group, a —NH-pyrimidinyl group or a —S-pyrimidinyl group; wherein the pyridinyl or pyrimidinyl groups are unsubstituted or substituted by one, two or three substituents selected from a —(CH₂)₀₋₃CN group, a —C(O)—(CH₂)₀₋₃—NR′R″ or a —(CH₂)₀₋₃NR′R″ group; and
iii) a group of formula (IIc), which group is selected from a —NH-purinyl group or a —S-purinyl group; wherein the purinyl group is unsubstituted or substituted by a —(CH₂)₀₋₃NR′R″ group; or
R₄ and R₅ together with the carbon atom to which they are attached form a pyrrolidinyl-purinyl group, wherein the purinyl group is unsubstituted or substituted by a —(CH₂)₀₋₃NR′R″ group; and
R′ and R″ each independently represent a hydrogen atom, a C₁-C₃ alkoxy group or a linear or branched C₁-C₃ alkyl group.

More preferably, the compound of formula (I) is one of
2-((6-Amino-9H-purin-9-yl)methyl)-5-chloro-3-o-tolylpyrrolo[1,2-f][1,2,4]triazin-4(3H)-one;
2-((6-Aminopyrimidin-4-ylamino)methyl)-5-chloro-3-o-tolylpyrrolo[1,2-f][1,2,4]triazin-4(3H)-one;
2-((6-Amino-9H-purin-9-yl)methyl)-5-cyclopropyl-3-o-tolylpyrrolo[1,2-f][1,2,4]triazin-4(3H)-one;
2-((6-amino-9H-purin-9-yl)methyl)-3-o-tolylpyrrolo[1,2-f][1,2,4]triazin-4(3H)-one;
2-((6-aminopyrimidin-4-ylamino)methyl)-3-o-tolylpyrrolo[1,2-f][1,2,4]triazin-4(3H)-one;
4-((4-Oxo-3-o-tolyl-3,4-dihydropyrrolo[1,2-f][1,2,4]triazin-2-yl)methylamino)picolinamide;
2-((2-aminopyridin-4-ylamino)methyl)-3-o-tolylpyrrolo[1,2-f][1,2,4]triazin-4(3H)-one;
2-((9H-purin-6-ylamino)methyl)-3-o-tolylpyrrolo[1,2-f][1,2,4]triazin-4(3H)-one;
2-((6-Amino-9H-purin-9-yl)methyl)-3-cyclohexylpyrrolo[1,2-f][1,2,4]triazin-4(3H)-one;
2-((6-amino-9H-purin-9-yl)methyl)-5-methyl-3-o-tolylpyrrolo[1,2-f][1,2,4]triazin-4(3H)-one;
2-((9H-purin-6-ylthio)methyl)-5-methyl-3-o-tolylpyrrolo[1,2-f][1,2,4]triazin-4(3H)-one;
2-((6-amino-9H-purin-9-yl)methyl)-6-methyl-3-o-tolylpyrrolo[1,2-f][1,2,4]triazin-4(3H)-one;
2-((9H-purin-6-ylthio)methyl)-6-methyl-3-o-tolylpyrrolo[1,2-f][1,2,4]triazin-4(3H)-one;
2-(1-(6-amino-9H-purin-9-yl)ethyl)-3-phenylpyrrolo[1,2-f][1,2,4]triazin-4(3H)-one;
(S)-2-(1-(9H-purin-6-ylamino)propyl)-3-phenylpyrrolo[1,2-f][1,2,4]triazin-4(3H)-one;
(S)-2-(1-(6-aminopyrimidin-4-ylamino)propyl)-3-phenylpyrrolo[1,2-f][1,2,4]triazin-4(3H)-one;
(S)-2-(1-(2-amino-9H-purin-6-ylamino)propyl)-3-phenylpyrrolo[1,2-f][1,2,4]triazin-4(3H)-one;
(S)-4-amino-6-(1-(4-oxo-3-phenyl-3,4-dihydropyrrolo[1,2-f][1,2,4]triazin-2-yl)propylamino)pyrimidine-5-carbonitrile;
(R)-2-(1-(9H-purin-6-ylamino)propyl)-3-phenylpyrrolo[1,2-f][1,2,4]triazin-4(3H)-one;
(S)-2-(1-(9H-purin-6-ylamino)ethyl)-3-phenylpyrrolo[1,2-f][1,2,4]triazin-4(3H)-one;
(S)-2-(1-(2-amino-9H-purin-6-ylamino)ethyl)-3-phenylpyrrolo[1,2-f][1,2,4]triazin-4(3H)-one;
(S)-2-(1-(6-aminopyrimidin-4-ylamino)ethyl)-3-phenylpyrrolo[1,2-f][1,2,4]triazin-4(3H)-one;
(S)-4-amino-6-(1-(4-oxo-3-phenyl-3,4-dihydropyrrolo[1,2-f][1,2,4]triazin-2-yl)ethylamino)pyrimidine-5-carbonitrile;
2-(1-(6-amino-9H-purin-9-yl)ethyl)-5-methyl-3-phenylpyrrolo[1,2-f][1,2,4]triazin-4(3H)-one;
2-((6-Amino-9H-purin-9-yl)methyl)-3-o-tolyl-5-(trifluoromethyl)pyrrolo[1,2-f][1,2,4]triazin-4(3H)-one;
2-((6-Amino-9H-purin-9-yl)methyl)-5-chloro-3-phenylpyrrolo[1,2-f][1,2,4]triazin-4(3H)-one;
2-((6-Amino-9H-purin-9-yl)methyl)-5-chloro-3-(3-methoxyphenyl)pyrrolo[1,2-f][1,2,4]triazin-4(3H)-one;
2-((6-Amino-9H-purin-9-yl)methyl)-5-chloro-3-(2,4-difluorophenyl)pyrrolo-[1,2-f][1,2,4]triazin-4(3H)-one;
2-((6-Amino-9H-purin-9-yl)methyl)-3-benzyl-5-chloropyrrolo[1,2-f][1,2,4]-triazin-4(3H)-one;
2-((6-amino-9H-purin-9-yl)methyl)-3-phenylimidazo[1,2-f][1,2,4]triazin-4(3H)-one;
2-((6-amino-9H-purin-9-yl)methyl)-3-o-tolylimidazo[1,2-f][1,2,4]triazin-4(3H)-one;
2-((6-Amino-9H-purin-9-yl)methyl)-5-chloro-3-(pyridin-4-yl)pyrrolo[1,2-f][1,2,4]triazin-4(3H)-one;
2-((6-Amino-9H-purin-9-yl)methyl)-5-chloro-3-(tetrahydro-2H-pyran-4-yl)pyrrolo-[1,2-f][1,2,4]triazin-4(3H)-one;
2-((6-Amino-9H-purin-9-yl)methyl)-5-chloro-3-(1-methylpiperidin-4-yl)pyrrolo[1,2-f][1,2,4]triazin-4(3H)-one;
(S)-2-(1-(9H-Purin-6-ylamino)ethyl)-3-(3-fluorophenyl)pyrrolo[1,2-f][1,2,4]triazin-4(3H)-one;
(S)-4-Amino-6-(1-(3-(3-fluorophenyl)-4-oxo-3,4-dihydropyrrolo[1,2-f][1,2,4]thazin-2-yl)ethylamino)pyrimidine-5-carbonitrile;
(S)-2-(1-(9H-Purin-6-ylamino)ethyl)-3-(3,5-difluorophenyl)pyrrolo[1,2-f][1,2,4]triazin-4(3H)-one;
(S)-4-Amino-6-(1-(3-(3,5-difluorophenyl)-4-oxo-3,4-dihydropyrrolo[1,2-f][1,2,4]thazin-2-yl)ethylamino)pyrimidine-5-carbonitrile;
2-((6-Amino-9H-purin-9-yl)methyl)-5-chloro-3-methylpyrrolo[1,2-f][1,2,4]triazin-4(3H)-one;
2-((6-Amino-9H-purin-9-yl)methyl)-3-((1r,4r)-4-aminocyclohexyl)-5-chloropyrrolo[1,2-f][1,2,4]triazin-4(3H)-one;
(R)-2-((6-Amino-9H-purin-9-yl)methyl)-5-chloro-3-(1-phenylethyl)pyrrolo[1,2-f][1,2,4]triazin-4(3H)-one;
(S)-2-((6-Amino-9H-purin-9-yl)methyl)-5-chloro-3-(1-phenylethyl)pyrrolo[1,2-f][1,2,4]triazin-4(3H)-one;
(S)-4-amino-6-(1-(4-oxo-3-(pyridin-2-yl)-3,4-dihydropyrrolo[1,2-f][1,2,4]triazin-2-yl)ethylamino)pyrimidine-5-carbonitrile;
(S)-2-(1-(9H-purin-6-yl)pyrrolidin-2-yl)-3-phenylpyrrolo[1,2-f][1,2,4]triazin-4(3H)-one;
(S)-4-amino-6-(1-(4-oxo-3-phenyl-5-(trifluoromethyl)-3,4-dihydropyrrolo[1,2-f][1,2,4]triazin-2-yl)ethylamino)pyrimidine-5-carbonitrile;
(S)-2-(1-(9H-purin-6-ylamino)ethyl)-3-phenyl-5-(trifluoromethyl)pyrrolo[1,2-f][1,2,4]triazin-4(3H)-one;
(S)-4-amino-6-(1-(5-(difluoromethyl)-4-oxo-3-phenyl-3,4-dihydropyrrolo[1,2-f][1,2,4]thazin-2-yl)ethylamino)pyrimidine-5-carbonitrile;
(S)-2-(1-(9H-purin-6-ylamino)ethyl)-5-(difluoromethyl)-3-phenylpyrrolo[1,2-f][1,2,4]triazin-4(3H)-one;
(S)-2-(1-(9H-purin-6-ylamino)ethyl)-3-phenylimidazo[1,2-f][1,2,4]triazin-4(3H)-one;
(S)-4-amino-6-(1-(4-oxo-3-phenyl-3,4-dihydroimidazo[1,2-f][1,2,4]triazin-2-yl)ethylamino)pyrimidine-5-carbonitrile;
2-(1-(9H-purin-6-ylamino)-3,3,3-trifluoropropyl)-3-phenylpyrrolo[1,2-f][1,2,4]triazin-4(3H)-one;
4-amino-6-(3,3,3-trifluoro-1-(4-oxo-3-phenyl-3,4-dihydropyrrolo[1,2-f][1,2,4]triazin-2-yl)propylamino)pyrimidine-5-carbonitrile;
(S)-4-amino-6-(2-(4-oxo-3-phenyl-3,4-dihydropyrrolo[1,2-f][1,2,4]triazin-2-yl)pyrrolidin-1-yl)pyrimidine-5-carbonitrile;
(S)-3-phenyl-2-(1-(pyrazolo[1,5-a]pyrimidin-7-ylamino)ethyl)pyrrolo[1,2-f][1,2,4]triazin-4(3H)-one;
2-((6-amino-9H-purin-9-yl)methyl)-5-(difluoromethyl)-3-phenylpyrrolo[1,2-f][1,2,4]triazin-4(3H)-one;
(S)-2-(1-(2-amino-9H-purin-6-yl)pyrrolidin-2-yl)-3-phenylpyrrolo[1,2-f][1,2,4]triazin-4(3H)-one;
(S)-2-(1-(4,6-diamino-1,3,5-triazin-2-ylamino)ethyl)-3-phenylpyrrolo[1,2-f][1,2,4]triazin-4(3H)-one;
(S)-2-((6-amino-9H-purin-9-yl)methyl)-5-chloro-3-(1-(5-fluoropyridin-2-yl)ethyl)pyrrolo[1,2-f][1,2,4]triazin-4(3H)-one;
(S)-2-(1-(2-amino-9H-purin-6-ylamino)ethyl)-3-(3,5-difluorophenyl)pyrrolo[1,2-f][1,2,4]triazin-4(3H)-one;
(S)-2-(1-(6-amino-5-cyanopyrimidin-4-ylamino)ethyl)-4-oxo-3-phenyl-3,4-dihydropyrrolo[1,2-f][1,2,4]thazine-5-carbonitrile;
(S)-2-(1-(9H-purin-6-ylamino)ethyl)-4-oxo-3-phenyl-3,4-dihydropyrrolo[1,2-f][1,2,4]triazine-5-carbonitrile;
(R)-2-(1-(9H-purin-6-ylamino)-2-hydroxyethyl)-3-phenylpyrrolo[1,2-f][1,2,4]triazin-4(3H)-one;
(R)-4-amino-6-(2-hydroxy-1-(4-oxo-3-phenyl-3,4-dihydropyrrolo[1,2-f][1,2,4]triazin-2-yl)ethylamino)pyrimidine-5-carbonitrile;
(S)-2-(1-(2-amino-9H-purin-6-ylamino)ethyl)-3-phenylimidazo[1,2-f][1,2,4]triazin-4(3H)-one;
(S)-2-(1-(7H-pyrrolo[2,3-d]pyrimidin-4-ylamino)ethyl)-3-phenylpyrrolo[1,2-f][1,2,4]triazin-4(3H)-one;
(S)-4-amino-6-(methyl(1-(4-oxo-3-phenyl-3,4-dihydropyrrolo[1,2-f][1,2,4]triazin-2-yl)ethyl)amino)pyrimidine-5-carbonitrile;
(S)-2-(1-(methyl(9H-purin-6-yl)amino)ethyl)-3-phenylpyrrolo[1,2-f][1,2,4]triazin-4(3H)-one;
(S)-2-(1-(9H-purin-6-ylamino)ethyl)-5-methyl-3-phenylpyrrolo[1,2-f][1,2,4]triazin-4(3H)-one;
(S)-4-amino-6-(1-(5-methyl-4-oxo-3-phenyl-3,4-dihydropyrrolo[1,2-f][1,2,4]triazin-2-yl)ethylamino)pyrimidine-5-carbonitrile;
(S)-2-(1-(9H-purin-6-ylamino)ethyl)-7-methyl-3-phenylpyrrolo[1,2-f][1,2,4]triazin-4(3H)-one;
(S)-4-amino-6-(1-(7-methyl-4-oxo-3-phenyl-3,4-dihydropyrrolo[1,2-f][1,2,4]triazin-2-yl)ethylamino)pyrimidine-5-carbonitrile;
(S)-2-(4,4-difluoro-1-(9H-purin-6-yl)pyrrolidin-2-yl)-3-phenylpyrrolo[1,2-f][1,2,4]triazin-4(3H)-one;
(S)-4-amino-6-(4,4-difluoro-2-(4-oxo-3-phenyl-3,4-dihydropyrrolo[1,2-f][1,2,4]triazin-2-yl)pyrrolidin-1-yl)pyrimidine-5-carbonitrile;
(S)-2-(1-(9H-purin-6-ylamino)ethyl)-6-fluoro-3-phenylpyrrolo[1,2-f][1,2,4]triazin-4(3H)-one;
(S)-4-amino-6-(1-(6-fluoro-4-oxo-3-phenyl-3,4-dihydropyrrolo[1,2-f][1,2,4]triazin-2-yl)ethylamino)pyrimidine-5-carbonitrile;
2-((S)-1-(9H-purin-6-ylamino)ethyl)-3-((S)-1-phenylethyl)pyrrolo[1,2-f][1,2,4]triazin-4(3H)-one;
4-amino-6-((S)-1-(4-oxo-3-((S)-1-phenylethyl)-3,4-dihydropyrrolo[1,2-f][1,2,4]triazin-2-yl)ethylamino)pyrimidine-5-carbonitrile;
(S)-4-amino-6-(1-(3-(2,6-dimethylphenyl)-4-oxo-3,4-dihydropyrrolo[1,2-f][1,2,4]triazin-2-yl)ethylamino)pyrimidine-5-carbonitrile;
(S)-2-((9H-purin-6-ylamino)methyl)-3-(1-phenylethyl)pyrrolo[1,2-f][1,2,4]triazin-4(3H)-one;
(S)-4-amino-6-((4-oxo-3-(1-phenylethyl)-3,4-dihydropyrrolo[1,2-f][1,2,4]triazin-2-yl)methylamino)pyrimidine-5-carbonitrile;
(S)-2-(1-(5-fluoro-7H-pyrrolo[2,3-d]pyrimidin-4-ylamino)ethyl)-3-phenylpyrrolo[1,2-f][1,2,4]triazin-4(3H)-one;
(S)-2-(1-(9H-purin-6-ylamino)ethyl)-3-(2,6-dimethylphenyl)pyrrolo[1,2-f][1,2,4]triazin-4(3H)-one;
(S)-4-amino-6-(1-(5-fluoro-4-oxo-3-phenyl-3,4-dihydropyrrolo[1,2-f][1,2,4]triazin-2-yl)ethylamino)pyrimidine-5-carbonitrile;
(S)-2-(1-(9H-purin-6-ylamino)ethyl)-5-fluoro-3-phenylpyrrolo[1,2-f][1,2,4]triazin-4(3H)-one;
(S)-2-(1-(6-amino-5-cyanopyrimidin-4-ylamino)ethyl)-3-(3,5-difluorophenyl)-4-oxo-3,4-dihydropyrrolo[1,2-f][1,2,4]triazine-5-carbonitrile;
(S)-4-amino-6-(1-(3-(3,5-difluorophenyl)-4-oxo-3,4-dihydroimidazo[1,2-f][1,2,4]triazin-2-yl)ethylamino)pyrimidine-5-carbonitrile;
(S)-2-(1-(9H-purin-6-ylamino)ethyl)-3-(3,5-difluorophenyl)-4-oxo-3,4-dihydropyrrolo[1,2-f][1,2,4]triazine-5-carbonitrile;
4-amino-6-((1S)-1-(5-(1,2-dihydroxyethyl)-4-oxo-3-phenyl-3,4-dihydropyrrolo[1,2-f][1,2,4]triazin-2-yl)ethylamino)pyrimidine-5-carbonitrile;
(S)-4-amino-6-(1-(3-(3,5-difluorophenyl)-4-oxo-5-(trifluoromethyl)-3,4-dihydropyrrolo[1,2-f][1,2,4]triazin-2-yl)ethylamino)pyrimidine-5-carbonitrile;
(S)-2-(1-(9H-Purin-6-ylamino)ethyl)-3-(3,5-difluorophenyl)-5-(trifluoromethyl)pyrrolo[1,2-f][1,2,4]thazin-4(3H)-one;
(S)-4-Amino-6-(1-(5-(hydroxymethyl)-4-oxo-3-phenyl-3,4-dihydropyrrolo[1,2-f][1,2,4]thazin-2-yl)ethylamino)pyrimidine-5-carbonitrile;
(S)-2-(1-(6-Amino-5-(trifluoromethyl)pyrimidin-4-ylamino)ethyl)-3-phenylpyrrolo[1,2-f][1,2,4]thazin-4(3H)-one;
(S)-2-(1-(6-Amino-5-cyanopyrimidin-4-ylamino)ethyl)-4-oxo-3-(pyridin-2-ylmethyl)-3,4-dihydropyrrolo[1,2-f][1,2,4]thazine-5-carbonitrile;
(S)-2-(1-(9H-Purin-6-ylamino)ethyl)-5-(difluoromethyl)-3-(3,5-difluorophenyl)pyrrolo[1,2-f][1,2,4]thazin-4(3H)-one;
(S)-2-(1-(9H-Purin-6-ylamino)ethyl)-3-(3,5-difluorophenyl)imidazo[1,2-f][1,2,4]thazin-4(3H)-one;
(S)-4-Amino-6-(1-(5-(difluoromethyl)-3-(3,5-difluorophenyl)-4-oxo-3,4-dihydropyrrolo[1,2-f][1,2,4]thazin-2-yl)ethylamino)pyrimidine-5-carbonitrile;
(S)-2-(1-(2-Amino-9H-purin-6-ylamino)ethyl)-5-(difluoromethyl)-3-(3,5-difluorophenyl)pyrrolo[1,2-f][1,2,4]thazin-4(3H)-one;
(S)-2-(1-(2-Amino-9H-purin-6-ylamino)ethyl)-3-(3,5-difluorophenyl)-4-oxo-3,4-dihydropyrrolo[1,2-f][1,2,4]thazine-5-carbonitrile;
2-(1-(9H-Purin-6-ylamino)-2,2,2-trifluoroethyl)-3-phenylpyrrolo[1,2-f][1,2,4]thazin-4(3H)-one;
(S)-4-Amino-6-(1-(3-benzyl-4-oxo-3,4-dihydropyrrolo[1,2-f][1,2,4]thazin-2-yl)ethylamino)pyrimidine-5-carbonitrile;
(S)-2-(1-(6-Amino-5-fluoropyrimidin-4-ylamino)ethyl)-3-phenylpyrrolo[1,2-f][1,2,4]thazin-4(3H)-one;
(S)-2-(1-(6-Amino-5-fluoropyrimidin-4-ylamino)ethyl)-5-(difluoromethyl)-3-(3,5-difluorophenyl)pyrrolo[1,2-f][1,2,4]thazin-4(3H)-one;
(S)-2-(1-(6-Amino-5-cyanopyrimidin-4-ylamino)propyl)-3-(3,5-difluorophenyl)-4-oxo-3,4-dihydropyrrolo[1,2-f][1,2,4]thazine-5-carbonitrile;
(S)-2-(1-(6-Amino-5-cyanopyrimidin-4-ylamino)ethyl)-3-(3,5-dichlorophenyl)-4-oxo-3,4-dihydropyrrolo[1,2-f][1,2,4]thazine-5-carbonitrile;
(S)-2-(1-(6-Amino-5-fluoropyrimidin-4-ylamino)ethyl)-3-(3,5-difluorophenyl)-4-oxo-3,4-dihydropyrrolo[1,2-f][1,2,4]thazine-5-carbonitrile;
(S)-2-(1-(6-Amino-5-(trifluoromethyl)pyrimidin-4-ylamino)ethyl)-4-oxo-3-phenyl-3,4-dihydropyrrolo[1,2-f][1,2,4]triazine-5-carbonitrile;
(R)-2-(1-(6-Amino-5-cyanopyrimidin-4-ylamino)-2-hydroxyethyl)-3-(3,5-difluorophenyl)-4-oxo-3,4-dihydropyrrolo[1,2-f][1,2,4]thazine-5-carbonitrile;
(S)-2-(1-(6-Amino-5-carbamoylpyrimidin-4-ylamino)ethyl)-3-(3,5-difluorophenyl)-4-oxo-3,4-dihydropyrrolo[1,2-f][1,2,4]thazine-5-carboxamide;
(S)-2-(1-(6-Amino-5-cyanopyrimidin-4-ylamino)ethyl)-3-(3,5-difluorophenyl)-4-oxo-3,4-dihydropyrrolo[1,2-f][1,2,4]thazine-5-carboxamide;
(S)-2-(1-(6-Amino-5-cyanopyrimidin-4-ylamino)ethyl)-3-(2-chlorobenzyl)-4-oxo-3,4-dihydropyrrolo[1,2-f][1,2,4]thazine-5-carbonitrile;
2-((S)-1-(6-amino-5-cyanopyrimidin-4-ylamino)ethyl)-4-oxo-3-((S)-tetrahydro-2H-pyran-3-yl)-3,4-dihydropyrrolo[1,2-f][1,2,4]triazine-5-carbonitrile;
(R)-4-Amino-6-(1-(3-(3,5-difluorophenyl)-4-oxo-5-(trifluoromethyl)-3,4-dihydropyrrolo[1,2-f][1,2,4]thazin-2-yl)-2-hydroxyethylamino)pyrimidine-5-carbonitrile;
(S)-2-(1-(2-Amino-5-fluoropyrimidin-4-ylamino)ethyl)-3-(3,5-difluorophenyl)-4-oxo-3,4-dihydropyrrolo[1,2-f][1,2,4]thazine-5-carbonitrile;
(S)-2-(1-(2-Amino-5-cyanopyrimidin-4-ylamino)ethyl)-3-(3,5-difluorophenyl)-4-oxo-3,4-dihydropyrrolo[1,2-f][1,2,4]thazine-5-carbonitrile;
((S)-2-(1-(9H-Purin-6-ylamino)ethyl)-3-(3,5-difluorophenyl)-5-(2H-tetrazol-5-yl)pyrrolo[1,2-f][1,2,4]triazin-4(3H)-one;
(S)-4-Amino-6-(1-(3-((5-methylisoxazol-3-yl)methyl)-4-oxo-3,4-dihydropyrrolo[1,2-f][1,2,4]triazin-2-yl)ethylamino)pyrimidine-5-carbonitrile;
(S)-4-Amino-6-(1-(4-oxo-3-phenyl-7-(trifluoromethyl)-3,4-dihydropyrrolo[1,2-f][1,2,4]triazin-2-yl)ethylamino)pyrimidine-5-carbonitrile;
(S)-2-(1-(6-Amino-5-cyanopyrimidin-4-ylamino)ethyl)-4-oxo-3-phenyl-3,4-dihydropyrrolo[1,2-f][1,2,4]triazine-7-carbonitrile;
(S)-2-(1-(6-Amino-5-cyanopyrimidin-4-ylamino)ethyl)-3-(1-(4-methoxybenzyl)-1H-pyrazol-4-yl)-4-oxo-3,4-dihydropyrrolo[1,2-f][1,2,4]triazine-5-carbonitrile;
(S)-4-amino-6-(1-(4-oxo-3-phenyl-5-(thiazol-2-yl)-3,4-dihydropyrrolo[1,2-f][1,2,4]triazin-2-yl)ethylamino)pyrimidine-5-carbonitrile;
(S)-2-(1-(2,6-Diamino-5-cyanopyrimidin-4-ylamino)ethyl)-3-(3,5-difluorophenyl)-4-oxo-3,4-dihydropyrrolo[1,2-f][1,2,4]thazine-5-carbonitrile;
(S)-4-Amino-6-(1-(5-(morpholinomethyl)-4-oxo-3-phenyl-3,4-dihydropyrrolo[1,2-f][1,2,4]triazin-2-yl)ethylamino)pyrimidine-5-carbonitrile;
2-((S)-1-(6-Amino-5-cyanopyrimidin-4-ylamino)ethyl)-4-oxo-3-((R)-1-phenylethyl)-3,4-dihydropyrrolo[1,2-f][1,2,4]triazine-5-carbonitrile;
(S)-4-Amino-6-(1-(4-oxo-3-(1H-pyrazol-4-yl)-3,4-dihydropyrrolo[1,2-f][1,2,4]triazin-2-yl)ethylamino)pyrimidine-5-carbonitrile;
(S)-2-(1-(9H-Purin-6-ylamino)ethyl)-3-(3,5-difluorophenyl)-5-(5-methyl-1,2,4-oxadiazol-3-yl)pyrrolo[1,2-f][1,2,4]triazin-4(3H)-one;
4-amino-6-((S)-1-(4-oxo-3-((S)-tetrahydro-2H-pyran-3-yl)-5-(trifluoromethyl)-3,4-dihydropyrrolo[1,2-f][1,2,4]triazin-2-yl)ethylamino)pyrimidine-5-carbonitrile;
(S)-4-Amino-6-(1-(3-(5-methyl-1H-pyrazol-3-yl)-4-oxo-3,4-dihydropyrrolo[1,2-f][1,2,4]triazin-2-yl)ethylamino)pyrimidine-5-carbonitrile;
(S)-2-(1-(6-Amino-5-cyanopyrimidin-4-ylamino)ethyl)-4-oxo-3-phenyl-3,4-dihydropyrrolo[1,2-f][1,2,4]triazine-5-carboxylic acid;
2-((S)-1-(6-amino-5-cyanopyrimidin-4-ylamino)ethyl)-4-oxo-3-((R)-tetrahydro-2H-pyran-3-yl)-3,4-dihydropyrrolo[1,2-f][1,2,4]triazine-5-carbonitrile;
(S)-4-Amino-6-(1-(3-(5-fluoropyridin-3-yl)-4-oxo-3,4-dihydropyrrolo[1,2-f][1,2,4]triazin-2-yl)ethylamino)pyrimidine-5-carbonitrile;
(S)-4-Amino-6-(1-(4-oxo-3-(1H-pyrazol-3-yl)-3,4-dihydropyrrolo[1,2-f][1,2,4]triazin-2-yl)ethylamino)pyrimidine-5-carbonitrile;
(S)-4-Amino-6-(1-(4-oxo-3-(pyrimidin-5-yl)-3,4-dihydropyrrolo[1,2-f][1,2,4]triazin-2-yl)ethylamino)pyrimidine-5-carbonitrile;
4-amino-6-((S)-1-(4-oxo-3-((R)-tetrahydro-2H-pyran-3-yl)-5-(trifluoromethyl)-3,4-dihydropyrrolo[1,2-f][1,2,4]triazin-2-yl)ethylamino)pyrimidine-5-carbonitrile;
(S)-2,4-Diamino-6-(1-(4-oxo-3-phenyl-3,4-dihydropyrrolo[1,2-f][1,2,4]triazin-2-yl)ethylamino)pyrimidine-5-carbonitrile;
(S)-4-(1-(3-((1H-Pyrazol-3-yl)methyl)-4-oxo-3,4-dihydropyrrolo[1,2-f][1,2,4]triazin-2-yl)ethylamino)-6-aminopyrimidine-5-carbonitrile;
(S)-4-Amino-6-(1-(4-oxo-3-(tetrahydro-2H-pyran-4-yl)-3,4-dihydropyrrolo[1,2-f][1,2,4]triazin-2-yl)ethylamino)pyrimidine-5-carbonitrile;
(S)-4-Amino-6-(1-(4-oxo-3-(2,2,2-trifluoroethyl)-3,4-dihydropyrrolo[1,2-f][1,2,4]triazin-2-yl)ethylamino)pyrimidine-5-carbonitrile;
(S)-4-Amino-6-(1-(3-cyclobutyl-4-oxo-3,4-dihydropyrrolo[1,2-f][1,2,4]triazin-2-yl)ethylamino)pyrimidine-5-carbonitrile;
(S)-2-Amino-4-(1-(4-oxo-3-phenyl-3,4-dihydropyrrolo[1,2-f][1,2,4]triazin-2-yl)ethylamino)pyrimidine-5-carbonitrile;
4-Amino-6-(1-(5-(1-methyl-1H-pyrazol-4-yl)-4-oxo-3-phenyl-3,4-dihydropyrrolo[1,2-f][1,2,4]triazin-2-yl)ethylamino)pyrimidine-5-carbonitrile;
(S)-4-Amino-6-(1-(3-cyclopropyl-4-oxo-3,4-dihydropyrrolo[1,2-f][1,2,4]triazin-2-yl)ethylamino)pyrimidine-5-carbonitrile;
(S)-4-Amino-6-(1-(5-bromo-4-oxo-3,4-dihydropyrrolo[1,2-f][1,2,4]triazin-2-yl)ethylamino)pyrimidine-5-carbonitrile;
4-amino-6-((S)-1-(4-oxo-3-((R)-tetrahydro-2H-pyran-3-yl)-3,4-dihydropyrrolo[1,2-f][1,2,4]triazin-2-yl)ethylamino)pyrimidine-5-carbonitrile;
(S)-4-Amino-6-(1-(5-bromo-4-oxo-3-phenyl-3,4-dihydropyrrolo[1,2-f][1,2,4]triazin-2-yl)ethylamino)pyrimidine-5-carbonitrile;
2-((3-Iodo-1H-pyrazolo[3,4-d]pyrimidin-4-ylamino)methyl)-5-methyl-3-o-tolylpyrrolo[1,2-f][1,2,4]triazin-4(3H)-one;
(S)-2-(1-(6-Amino-5-cyanopyrimidin-4-ylamino)ethyl)-3-(5-fluoropyridin-3-yl)-4-oxo-3,4-dihydropyrrolo[1,2-f][1,2,4]triazine-5-carbonitrile;
4-amino-6-((S)-1-(4-oxo-3-((S)-tetrahydro-2H-pyran-3-yl)-3,4-dihydropyrrolo[1,2-f][1,2,4]triazin-2-yl)ethylamino)pyrimidine-5-carbonitrile;
(S)-4-Amino-6-(1-(4-oxo-3-phenyl-5-(1H-pyrazol-4-yl)-3,4-dihydropyrrolo[1,2-f][1,2,4]triazin-2-yl)ethylamino)pyrimidine-5-carbonitrile;
(S)-4-Amino-6-(1-(3-(isoxazol-3-yl)-4-oxo-3,4-dihydropyrrolo[1,2-f][1,2,4]triazin-2-yl)ethylamino)pyrimidine-5-carbonitrile;
(S)-2-(1-(6-Amino-5-cyanopyrimidin-4-ylamino)ethyl)-N,N-dimethyl-4-oxo-3-phenyl-3,4-dihydropyrrolo[1,2-f][1,2,4]triazine-5-carboxamide;
(S)-4-Amino-6-(1-(3-(1-methyl-1H-pyrazol-3-yl)-4-oxo-3,4-dihydropyrrolo[1,2-f][1,2,4]triazin-2-yl)ethylamino)pyrimidine-5-carbonitrile
(S)-2-(1-(6-Amino-5-cyanopyrimidin-4-ylamino)ethyl)-4-oxo-3-phenyl-N-propyl-3,4-dihydropyrrolo[1,2-f][1,2,4]triazine-5-carboxamide;
2-((S)-1-(9H-Purin-6-ylamino)ethyl)-3-(tetrahydro-2H-pyran-3-yl)pyrrolo[1,2-f][1,2,4]triazin-4(3H)-one;
2-((S)-1-(9H-purin-6-ylamino)ethyl)-3-((S)-tetrahydro-2H-pyran-3-yl)pyrrolo[1,2-f][1,2,4]triazin-4(3H)-one;
(S)-4-Amino-6-(3-hydroxy-1-(4-oxo-3-phenyl-3,4-dihydropyrrolo[1,2-f][1,2,4]triazin-2-yl)propylamino)pyrimidine-5-carbonitrile;
(S)-2-(1-(9H-Purin-6-ylamino)-3-hydroxypropyl)-3-phenylpyrrolo[1,2-f][1,2,4]triazin-4(3H)-one;
(R)-4-Amino-6-(1-(3-(3,5-difluorophenyl)-4-oxo-3,4-dihydropyrrolo[1,2-f][1,2,4]triazin-2-yl)-2-hydroxyethylamino)pyrimidine-5-carbonitrile;
4-Amino-6-((4-oxo-3-o-tolyl-3,4-dihydropyrrolo[1,2-f][1,2,4]triazin-2-yl)methylamino)pyrimidine-5-carbonitrile;
(S)-4-Amino-6-(1-(5-(2-hydroxyethyl)-4-oxo-3-phenyl-3,4-dihydropyrrolo[1,2-f][1,2,4]triazin-2-yl)ethylamino)pyrimidine-5-carbonitrile;
S)-2-(1-(6-amino-5-cyanopyrimidin-4-ylamino)-3-hydroxypropyl)-3-(3,5-difluorophenyl)-4-oxo-3,4-dihydropyrrolo[1,2-f][1,2,4]triazine-5-carbonitrile;
(S)-2-(1-(9H-Purin-6-ylamino)ethyl)-3-(5-fluoropyridin-3-yl)-4-oxo-3,4-dihydropyrrolo[1,2-f][1,2,4]triazine-5-carbonitrile;
(S)-4-Amino-6-(1-(5-(2-methyloxazol-5-yl)-4-oxo-3-phenyl-3,4-dihydropyrrolo[1,2-f][1,2,4]triazin-2-yl)ethylamino)pyrimidine-5-carbonitrile;
(S)-4-Amino-6-(1-(5-(2-methoxyethyl)-4-oxo-3-phenyl-3,4-dihydropyrrolo[1,2-f][1,2,4]triazin-2-yl)ethylamino)pyrimidine-5-carbonitrile;
(S)-Propyl 2-(1-(6-amino-5-cyanopyrimidin-4-ylamino)ethyl)-4-oxo-3-phenyl-3,4-dihydropyrrolo[1,2-f][1,2,4]triazine-5-carboxylate;
(S)-4-Amino-6-(3-hydroxy-1-(4-oxo-3,4-dihydropyrrolo[1,2-f][1,2,4]triazin-2-yl)propylamino)pyrimidine-5-carbonitrile;
(S)-2-(1-(9H-Purin-6-ylamino)-3-hydroxypropyl)pyrrolo[1,2-f][1,2,4]triazin-4(3H)-one;
(S)-4-amino-6-(1-(3-(3,5-difluorophenyl)-4-oxo-5-(trifluoromethyl)-3,4-dihydropyrrolo[1,2-f][1,2,4]triazin-2-yl)-3-hydroxypropylamino)pyrimidine-5-carbonitrile;
(S)-4-Amino-6-(1-(4-oxo-3-(6-(trifluoromethyl)pyridin-2-yl)-3,4-dihydropyrrolo[1,2-f][1,2,4]triazin-2-yl)ethylamino)pyrimidine-5-carbonitrile;
(S)-4-Amino-6-(1-(5-bromo-4-oxo-3-(3-(trifluoromethyl)phenyl)-3,4-dihydropyrrolo[1,2-f][1,2,4]triazin-2-yl)ethylamino)pyrimidine-5-carbonitrile;
(S)-2-(2-(1-(6-Amino-5-cyanopyrimidin-4-ylamino)ethyl)-4-oxo-3-phenyl-3,4-dihydropyrrolo[1,2-f][1,2,4]triazin-5-yl)ethyl acetate;
(S)-2-(1-(9H-Purin-6-ylamino)ethyl)-3-(6-(trifluoromethyl)pyridin-2-yl)pyrrolo[1,2-f][1,2,4]triazin-4(3H)-one;
2-((2S,4R)-1-(6-Amino-5-cyanopyrimidin-4-yl)-4-hydroxypyrrolidin-2-yl)-3-(3,5-difluorophenyl)-4-oxo-3,4-dihydropyrrolo[1,2-f][1,2,4]triazine-5-carbonitrile;
4-Amino-6-((2S,4R)-2-(5-(aminomethyl)-3-(3,5-difluorophenyl)-4-oxo-3,4-dihydropyrrolo[1,2-f][1,2,4]thazin-2-yl)-4-hydroxypyrrolidin-1-yl)pyrimidine-5-carbonitrile;
(S)-4-Amino-6-(1-(5-(4-methyl-1H-imidazol-1-yl)-4-oxo-3-phenyl-3,4-dihydropyrrolo[1,2-f][1,2,4]triazin-2-yl)ethylamino)pyrimidine-5-carbonitrile;
(S)-4-Amino-6-(1-(5-bromo-3-(3-methoxyphenyl)-4-oxo-3,4-dihydropyrrolo[1,2-f][1,2,4]triazin-2-yl)ethylamino)pyrimidine-5-carbonitrile;
(S)-2-(1-(6-Amino-5-cyanopyrimidin-4-ylamino)ethyl)-4-oxo-3-(3-(trifluoromethyl)phenyl)-3,4-dihydropyrrolo[1,2-f][1,2,4]triazine-5-carbonitrile;
(S)-4-Amino-6-(1-(5-bromo-3-(3-hydroxyphenyl)-4-oxo-3,4-dihydropyrrolo[1,2-f][1,2,4]thazin-2-yl)ethylamino)pyrimidine-5-carbonitrile;
(S)-4-Amino-6-(1-(3-(3-methoxyphenyl)-4-oxo-3,4-dihydropyrrolo[1,2-f][1,2,4]triazin-2-yl)ethylamino)pyrimidine-5-carbonitrile;
(S)-4-Amino-6-(1-(3-(3-hydroxyphenyl)-4-oxo-3,4-dihydropyrrolo[1,2-f][1,2,4]thazin-2-yl)ethylamino)pyrimidine-5-carbonitrile;
(S)-2-(1-(6-Amino-5-cyanopyrimidin-4-ylamino)ethyl)-3-(3-methoxyphenyl)-4-oxo-3,4-dihydropyrrolo[1,2-f][1,2,4]thazine-5-carbonitrile;
4-Amino-6-(1-(4-oxo-3-phenyl-3,4-dihydropyrrolo[1,2-f][1,2,4]triazin-2-yl)cyclopropylamino)pyrimidine-5-carbonitrile;
2-(1-(9H-Purin-6-ylamino)cyclopropyl)-3-phenylpyrrolo[1,2-f][1,2,4]triazin-4(3H)-one;
(S)-4-Amino-6-(1-(4-oxo-3-(3-(trifluoromethyl)phenyl)-3,4-dihydropyrrolo[1,2-f][1,2,4]triazin-2-yl)ethylamino)pyrimidine-5-carbonitrile;
(S)-2-(1-(6-Amino-5-cyanopyrimidin-4-ylamino)ethyl)-3-(3-hydroxyphenyl)-4-oxo-3,4-dihydropyrrolo[1,2-f][1,2,4]thazine-5-carbonitrile;
(S)-2-(1-(9H-purin-6-ylamino)ethyl)-3-(pyridin-2-yl)pyrrolo[1,2-f][1,2,4]triazin-4(3H)-one;
(S)-2-(1-(9H-purin-6-ylamino)propyl)-3-phenylimidazo[1,2-f][1,2,4]triazin-4(3H)-one; and
(S)-4-amino-6-(1-(4-oxo-3-phenyl-3,4-dihydroimidazo[1,2-f][1,2,4]triazin-2-yl)propylamino)pyrimidine-5-carbonitrile.

Further preferred inhibitors of phosphoinositide 3-kinase delta may be selected from nortriptyline, idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, XL-499, KAR-4141, RP-5090, PWT-143, IPI-443, RP-6503, ONO-146040, SPR-965, LOR-220, SF-2626, X-339, X-480, PQR-401, INCB-050465, LS-008, CLR-457, PCN-5603, 7-hydroxystaurosporine, PF-04691502, TG-100115, BGT-226, SF-1126, PKI-179 and panulisib, for instance idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, XL-499, KAR-4141, RP-5090, PWT-143, IPI-443, RP-6503, ONO-146040, SPR-965, LOR-220, SF-2626, X-339, X-480, PQR-401, INCB-050465, LS-008, CLR-457, PCN-5603, 7-hydroxystaurosporine, PF-04691502, TG-100115, BGT-226, SF-1126, PKI-179 and panulisib.

Typically, inhibitors of phosphoinositide 3-kinase delta for use in accordance with the present invention are selected from the group consisting of nortriptyline, idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, LAS191954, XL-499, KAR-4141, RP-5090, PWT-143, IPI-443, RP-6503, ONO-146040, SPR-965, LOR-220, SF-2626, X-339, X-480, PQR-401, INCB-050465, LS-008, CLR-457, PCN-5603, 7-hydroxystaurosporine, PF-04691502, TG-100115, BGT-226, SF-1126, PKI-179 and panulisib, for instance idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, LAS191954, XL-499, KAR-4141, RP-5090, PWT-143, IPI-443, RP-6503, ONO-146040, SPR-965, LOR-220, SF-2626, X-339, X-480, PQR-401, INCB-050465, LS-008, CLR-457, PCN-5603, 7-hydroxystaurosporine, PF-04691502, TG-100115, BGT-226, SF-1126, PKI-179 and panulisib.

Preferably, inhibitors of phosphoinositide 3-kinase delta for use in accordance with the present invention are selected from the group consisting of nortriptyline, idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, LAS191954, XL-499, KAR-4141, RP-5090, PWT-143, IPI-443, RP-6503, ONO-146040, SPR-965, LOR-220, SF-2626, X-339, X-480, PQR-401, INCB-050465, LS-008 and CLR-457, for instance idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, LAS191954, XL-499, KAR-4141, RP-5090, PWT-143, IPI-443, RP-6503, ONO-146040, SPR-965, LOR-220, SF-2626, X-339, X-480, PQR-401, INCB-050465, LS-008 and CLR-457.

More preferably, inhibitors of phosphoinositide 3-kinase delta for use in accordance with the present invention are selected from the group consisting of nortriptyline, idelalisib, duvelisib, enzastaurin, rigosertib, GSK-2269557, UCB-5857, RV-1729, RP-6530, LAS191954, XL-499, KAR-4141, RP-5090, PWT-143, IPI-443 and RP-6503, for instance idelalisib, duvelisib, enzastaurin, rigosertib, GSK-2269557, UCB-5857, RV-1729, RP-6530, LAS191954, XL-499, KAR-4141, RP-5090, PWT-143, IPI-443 and RP-6503.

Still more preferably, inhibitors of phosphoinositide 3-kinase delta for use in accordance with the present invention are selected from the group consisting of idelalisib, duvelisib, UCB-5857, RP-6530, LAS191954, XL-499, KAR-4141, RP-5090, PWT-143, IPI-443 and RP-6503.

Alternatively, inhibitors of phosphoinositide 3-kinase delta for use in accordance with the present invention are selected from the group consisting of LAS191954, alpelisib ((S)—N1-(4-methyl-5-(2-(1,1,1-trifluoro-2-methylpropan-2-yl)pyridin-4-yl)thiazol-2-yl)pyrrolidine-1,2-dicarboxamide), duvelisib ((S)-3-(1-((9H-purin-6-yl)amino)ethyl)-8-chloro-2-phenylisoquinolin-1(2H)-one), rigosertib sodium (sodium (E)-2-((2-methoxy-5-(((2,4,6-trimethoxystyryl)sulfonyl)methyl)phenyl)amino)acetate), and 6-(2-((4-amino-3-(3-hydroxyphenyl)-1H-pyrazolo[3,4-d]pyrimidin-1-yl)methyl)-3-(2-chlorobenzyl)-4-oxo-3,4-dihydroquinazolin-5-yl)-N, N-bis(2-methoxyethyl)hex-5-ynamide.

Most preferably, the inhibitor of phosphoinositide 3-kinase delta for use in accordance with the present invention is LAS191954.

, LAS191954, which has the structure of formula (A) and corresponds to (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile, as well as a process for its manufacture, is described in the International Patent Application No. WO 2012/146666.

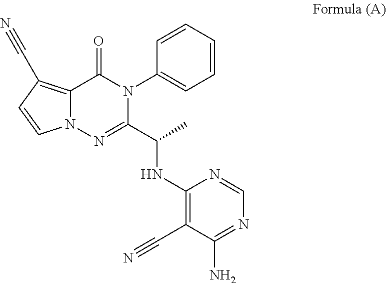 [SMILES: C[C@H](Nc1ncnc(N)c1C#N)c1nn2ccc(C#N)c2c(=O)n1-c1ccccc1]

In a preferred embodiment, the compound is a pharmaceutically acceptable crystalline addition salt of LAS191954 with a sulfonic acid derivative selected from methanesulfonic acid, naphthalene-2-sulfonic acid and para-toluenesulfonic acid, or a pharmaceutically acceptable solvate thereof.

In a particular embodiment, the compound is LAS191954methanesulfonate, or a pharmaceutically acceptable solvate thereof.

Typically, methanesulfonic acid (CAS RN 75-75-2) is a colourless liquid with the molecular formula CH₄O₃S (molecular weight of 96.11 g/mol). Salts of methanesulfonic acid are known as methanesulfonates, mesilates (International Nonproprietary Name or INN) or mesylates (United States Adopted Name or USAN).

In another particular embodiment, the compound is (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile naphthalene-2-sulfonate, or a pharmaceutically acceptable solvate thereof.

Typically, naphthalene-2-sulfonic acid (CAS RN 120-18-3) is a solid at 20° C. with the molecular formula C₁₀H₈O₃S (molecular weight of 208.24 g/mol). Salts of naphthalene-2-sulfonic acid are known as naphthalene-2-sulfonates, napsilates (INN) or napsylates (USAN).

In another particular embodiment, the compound is (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile para-toluenesulfonate, or a pharmaceutically acceptable solvate thereof.

Typically, para-toluenesulfonic acid (CAS RN 104-15-4) or tosylic acid is a solid at 20° C. with the molecular formula C₇H₈O₃S (molecular weight of 172.20 g/mol). Salts of para-toluenesulfonic acid are known as para-toluenesulfonates, tosilates (INN) or tosylates (USAN).

In still another particular embodiment, the compound is LAS191954 para-toluenesulfonate monohydrate.

Compounds for use in the present invention are typically commercially available or may be prepared in accordance with known methods.

Examples of suitable corticosteroids to be used in the combinations of the invention are selected form prednisolone, methylprednisolone, dexamethasone, dexamethasone cipecilate, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone furoate, rimexolone, prednisolone farnesylate, ciclesonide, butixocort propionate, deprodone propionate, fluticasone propionate, fluticasone furoate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate and hydrocortisone probutate.

Corticosteroids chosen from prednisolone, betamethasone, dexamethasone, and methylprednisolone are more preferred.

Methylprednisolone and prednisolone are particularly preferable, prednisolone being most preferable.

Any reference to corticosteroids within the scope of the present invention includes a reference to salts or derivatives thereof which may be formed from the corticosteroids. Examples of possible salts or derivatives include: sodium salts, sulphobenzoates, phosphates, isonicotinates, acetates, propionates, dihydrogen phosphates, palmitates, pivaiates, farnesylates, aceponates, suleptanates, prednicarbates, furoates or acetonides. In some cases the corticosteroids may also occur in the form of their hydrates.

The combinations of the invention may contain one or more other therapeutic agents. The other therapeutic agent is typically useful in the treatment or prevention of a skin disease as defined herein, preferably an immunobullous skin disease mediated by autoantibodies as defined herein.

The other therapeutic agent may be chosen from the group consisting of:
a) Immunosuppressants, such as Imuran (azathioprine), cyclophosphamide, sirolimus or Purinethol (6-mercaptopurine or 6-MP);
b) Anti-CD20 (lymphocyte protein) monoclonal antibodies such as Rituximab, Ocrelizumab, Ofatumumab or TRU-015;
c) Anti-CD52 (lymphocyte protein) monoclonal antibodies such as alemtuzumab;
d) Anti-CD25 (lymphocyte protein) such as daclizumab;
e) Anti-CD88 (lymphocyte protein), such as eculizumab or pexilizumab;
f) Anti-Interleukin 6 Receptor (IL-6R), such as tocilizumab;
g) Anti-Interleukin 12 Receptor (IL-12R)/Interleukin 23 Receptor (IL-23R), such as ustekinumab;
h) Anti-BAFF/BlyS such as belimumab, tabalumab, or blisibimod
i) Anti-TACl such as atacicept
j) Anti-BAFF receptor such as VAY736
k) Anti-CD19 such as MEDI-551
l) Anti-ICOSL such as AMG-557
m) Anti-FasL monoclonal antibodies
n) Btk inhibitors like ibrutinib
o) Calcineurin inhibitors such as cyclosporin A, pimecrolimus or tacrolimus;
p) Dyhydrofolate reductase inhibitors, such as Methotrexate or CH-1504;
q) Dihydroorotate dehydrogenase (DHODH) inhibitors such as leflunomide or teriflunomide;
r) Immunomodulators such as Glatiramer acetate (Copaxone), Laquinimod or Imiquimod;
s) Inhibitors of DNA synthesis and repair, such as Mitoxantrone or Cladribine;
t) Anti-alpha 4 integrin antibodies, such as Natalizumab (Tysabri);
u) Alpha 4 integrin antagonists such as R-1295, TBC-4746, CDP-323, ELND-002, Firategrast or TMC-2003;
v) Fumaric acid esters, such as dimethyl fumarate;
w) Anti-tumor necrosis factor-alpha (Anti-TNF-alpha) monoclonal antibodies such as Infliximab, Adalimumab or Certolizumab pegol;
x) Soluble Tumor necrosis factor-alpha (TNF-alpha) Antagonists such as Etanercept;
y) Inosine-monophosphate dehydrogenase (IMPDH) inhibitors, such as mycophenolate mophetyl, ribavirin, mizoribine or mycophenolic acid;
z) Cannabinoid receptor agonists such as Sativex;
aa) Chemokine CCR1 antagonists such as MLN-3897 or PS-031291;
bb) Chemokine CCR2 antagonists such as INCB-8696;
cc) Nuclear factor-kappaB (NF-kappaB or NFKB) Activation Inhibitors such as
Sulfasalazine, Iguratimod or MLN-0415;
dd) Adenosine A_{2A} agonists, such as ATL-313, ATL-146, CGS-21680, Regadenoson or UK-432,097;
ee) Sphingosine-1 (S1P) phosphate receptor agonists such as fingolimod, BAF-312, or ACT128800;
ff) Sphingosine-1 (S1P) liase inhibitors such as LX2931;
gg) Spleen tyrosine kinase (Syk) inhibitors, such as R-112;
hh) Protein Kinase Inhibitors (PKC) inhibitors, such as NVP-AEB071;
ii) Histamine 1 (H1) receptor antagonists, such as azelastine or ebastine;
jj) Mast cell stabilizers, such as nedocromil or chromoglycate;
kk) Chemoattractant receptor homologous molecule expressed on TH2 cells (CRTH2) inhibitors, such as OC-459, AZD-1981, ACT-129968, QAV-680; II) Vitamin D derivatives like calcipotriol (Daivonex);
mm) Anti-inflammatory agents, such as non-steroidal anti-inflammatory drugs (NSAIDs) or selective cyclooxygenase-2 (COX-2) inhibitors such as aceclofenac, diclofenac, ibuprofen, naproxen, apricoxib, celecoxib, cimicoxib, deracoxib, etoricoxib, lumiracoxib, parecoxib sodium, rofecoxib, selenocoxib-1 or valdecoxib;
nn) Anti-viral agents, such as aciclovir or tenofovir;
oo) Phosphodiestearase (PDE) Ill inhibitors;
pp) Phosphosdiesterase (PDE) IV inhibitors such as roflumilast or GRC-4039;
qq) Dual Phosphodiestearase (PDE) III/IV inhibitors;
rr) p38 Mitogen-Activated Protein Kinase (p38 MAPK) Inhibitors such as ARRY-797;
ss) Mitogen-activated extracellular signal regulated kinase kinase (MEK) inhibitor, such as ARRY-142886 or ARRY-438162;
tt) Janus kinase (JAK) inhibitors, such as tofacitinib (previously known as tasocitinib or CP-690,550) or INCB-18424;
uu) Interferons comprising Interferon beta 1a such as Avonex from Biogen Idec, CinnoVex from CinnaGen and Rebif from EMD Serono, and Interferon beta 1 b such as Betaferon from Schering and Betaseron from Berlex;
vv) Interferon alpha such as Sumiferon MP;
ww) Epidermal Growth Factor Receptor (EGFR) inhibitors such as erlotinib, Trastuzumab, Herceptin, Avastin, Platins (cisplatin, carboplatin) or Temazolamide;
xx) Antineoplastic agents such as Docetaxel, Estramustine, Anthracyclines, (doxorubicin (Adriamycin), epirubicin (Ellence), and liposomal doxorubicin (Doxil)), Taxanes (docetaxel (Taxotere), paclitaxel (Taxol), and protein-bound paclitaxel (Abraxane)), Cyclophosphamide (Cytoxan), Capecitabine (Xeloda), 5-fluorouracil (5-FU), Gemcitabine (Gemzar) or Vinorelbine (Navelbine);
yy) Tetracyclines, such as methacycline, doxycycline or minocycline;
zz) Analgesics, such as paracetamol;
aaa) Opioids such as, morphine, tramadol, oxycodone or fentanyl;
bbb) Kappa opioid agonists, such as nalfurafine, nalbuphine or ketazocine;
ccc) Neurokinin receptor 1 antagonists, such as aprepitant or fosaprepitant; or
ddd) Dihydropteroate synthase inhibitors, such as dapsone.

The other therapeutic agent is preferably chosen from
a) Dihydrofolate reductase inhibitors, such as Methotrexate or CH-1504;
b) Immunosuppressants, such as Imuran (azathioprine), cyclophosphamide, sirolimus or Purinethol (6-mercaptopurine or 6-MP);
c) Anti-tumor necrosis factor-alpha (Anti-TNF-alpha) monoclonal antibodies such as Infliximab, Adalimumab or Certolizumab pegol;
d) Soluble Tumor necrosis factor-alpha (TNF-alpha) Antagonists such as Etanercept;
e) Anti-CD20 (lymphocyte protein) monoclonal antibodies such as Rituximab, Ocrelizumab, Ofatumumab or TRU-015
f) Anti-BAFF/BlyS such as belimumab, tabalumab, or blisibimod
g) Anti-TACl such as atacicept
h) Anti-BAFF receptor such as VAY736
i) Anti-CD19 such as MEDI-551
j) Anti-ICOSL such as AMG-557
k) Anti-FasL monoclonal antibodies
l) Btk inhibitors like ibrutinib
m) Calcineurin inhibitors such as cyclosporine A, pimecrolimus or tacrolimus;
n) Inosine-monophosphate dehydrogenase (IMPDH) inhibitors, such as mycophenolate mophetyl, ribavirin, mizoribine or mycophenolic acid;
o) Tetracyclines, such as methacycline, doxycycline or minocycline; and
p) Dihydropteroate synthase inhibitors, such as dapsone;

The therapeutic agent is more preferably chosen from azathioprine, mizoribine, mycophenolate mofetil, mycophenolic acid, dapsone, acitretin, cyclophosphamide, immunoglobulin (Ig), thalidomide, tetracycline and rituximab.

In certain circumstances, the most preferable other therapeutic agent is azathioprine.

The skin diseases which may be treated in accordance with the present invention include psoriasis, atopic dermatitis (atopic eczema), contact dermatitis, seborrheic dermatitis, xerotic eczema, scalp eczema, hand eczema, dyshidrosis, discoid eczema, venous eczema, dermatitis herpetiformis, neurodermatitis, autoeczematization, acne, rosacea, cutaneous T-cell lymphomas (CTLC) such as mycosis fungoides (MF) and Sézare syndrome (SS), Hailey-Hailey disease, Epidermolysis Bullosa (EB) including but not limited to Epidermolysis Bullosa Simple (EBS), EBS Köbner variant, EBS of the hands and feet, EBS with anodontia/hypodontia, EBS herpetiformis, EBS with mottled hyperpigmentation, EBS Ogna variant, EBS superficialis, EBS with muscular dystrophy, EBS Mendes da Costa variant, lethal acantholytic EBS, EBS with plakophilin deficiency, EBS Dowling-Meara variant, EBS with pyloric atresia, migratory circinate EBS, and autosomal recessive EBS; Junctional Epidermolysis Bullosa including junctional EB Gravis variant, junctional EB with pyloric stenosis, generalized atrophic benign EB, cicatricial junctional EB, junctional EB progressive variant and junctional EB inversa, junctional EB Herlitz variant, and junctional EB with pyloric atresia; Dystrophic Epidermolysis Bullosa (DEB) including autosomal dominant DEB (such as generalized dominant DEB, acral dominant DEB, pretibial dominant DEB, pruriginosa dominant DEB, nails only dominant DEB, bullous dermolysis of the new born-dominant DEB), and autosomal recessive DEB (such as severe generalized recessive DEB, other than generalized recessive DEB, inverse recessive DEB, pruriginosa recessive DEB, centripetalis recessive DEB and bullous dermolysis of the new born-recessive DEB); Kindler Syndrome; Pemphigus Diseases, including but not limited to pemphigus vulgaris, pemphigus herpetiformis, pemphigus vegetans (including pemphigus vegetans Neumann type and pemphigus vegetans Hallopeua type), pemphigus foliaceus, Brazilian pemphigus (endemic pemphigus foliaceus), pemphigus erythematosus, immunoglobulin A (IgA) pemphigus (intercellular IgA dermatosis) and paraneoplastic pemphigus; Pemphigoid Diseases, including but not limited to bullous pemphigoid, pemphigoid gestationis (herpes gestationis), cicatricial pemphigoid, mucous membrane pemphigoid, linear IgA bullous disease (chronic bullous disease of childhood and adult linear IgA disease), acquired epidermolysis bullosa, dermatitis herpetiformis and lichen planus pemphigoides; bullous impetigo, scalded skin syndrome, miliaria crystalline, subcorneal pustular dermatosis, acute dermatitis, pompholyx, viral infections, transient acantholytic dermatosis (Grover's disease), porphyria cutanea tarda, blisters in diabetes and renal disease, bullous lupus erythematosus and toxic epidermal necrolysis (Lyell's disease).

Preferably, the skin disease is an immunobullous skin diseases mediated by autoantibodies.

The immunobullous skin diseases which may be treated in accordance with the present invention are characterized by pathogenic autoantibodies directed at antigens whose function is either cell-to-cell adhesion within the epidermis or adhesion of stratified squamous epithelium to dermis or mesenchyme. These target antigens are components of desmosomes or the functional unit of the basement membrane zone known as the adhesion complex (see Rook's Textbook of Dermatology, Wiley-Blackwell, Chapter 40-Immunobullous diseases).

Typically, the immunobullous skin disease is mediated by anti-Dsg autoantibodies. Preferably, the immunobullous skin disease is mediated by anti-Dsg1 and/or anti-Dsg3 autoantibodies. More preferably, the immunobullous skin disease is mediated by anti-Dsg3 autoantibodies.

Typically, the immunobullous skin disease is mediated by anti-dsDNA autoantibodies.

The immunobullous skin disease may be mediated by both anti-dsDNA autoantibodies and anti-Dsg autoantibodies as defined above.

Immunobullous skin diseases mediated by autoantibodies which may be treated in accordance with the invention include (but are not limited to):
Intraepidermal immunobullous diseases, such as pemphigus vulgaris, pemphigus vegetans, pemphigus foliaceus, endemic pemphigus foliaceus, intercellular IgA dermatosis, paraneoplastic pemphigus; and
Subepidermal immunobullous diseases, such bullous pemphigoid, mucous membrane pemphigoid, pemphigoid gestationis, linear IgA disease, epidermolysis bullosa acquisita, bullous systemic lupus erythematosus and dermatitis herpetiformis.

Typically, the immunobullous skin disease mediated by autoantibodies is pemphigus vulgaris, pemphigus vegetans, pemphigus foliaceus, endemic pemphigus foliaceus, paraneoplastic pemphigus or epidermolysis bullosa acquisita.

Usually, the immunobullous skin disease mediated by autoantibodies is pemphigus vulgaris, pemphigus vegetans, pemphigus foliaceus, endemic pemphigus foliaceus, or paraneoplastic pemphigus.

In some circumstances, the immunobullous skin disease mediated by autoantibodies is pemphigus vulgaris, pemphigus foliaceus or epidermolysis bullosa acquisita.

Preferably, the immunobullous skin disease mediated by autoantibodies is pemphigus vulgaris or pemphigus foliaceus.

More preferably, the immunobullous skin disease mediated by autoantibodies is pemphigus vulgaris or epidermolysis bullosa acquisita.

Most preferably, the immunobullous skin disease mediated by autoantibodies is pemphigus vulgaris.

In a preferred embodiment, the compound (a) is LAS191954 or a pharmaceutically acceptable salt and/or solvate thereof and the immunobullous skin disease mediated by autoantibodies treated is pemphigus vulgaris.

In a more preferred embodiment, the compound (a) is LAS191954 methanesulfonate, or a pharmaceutically acceptable solvate thereof and the immunobullous skin disease mediated by autoantibodies treated is pemphigus vulgaris.

In another more preferred embodiment, the compound (a) is LAS191954 naphthalene-2-sulfonate, or a pharmaceutically acceptable solvate thereof and the immunobullous skin disease mediated by autoantibodies treated is pemphigus vulgaris.

In another more preferred embodiment, the compound (a) is LAS191954 para-toluenesulfonate, or a pharmaceutically acceptable solvate thereof and the immunobullous skin disease mediated by autoantibodies treated is pemphigus vulgaris.

In another more preferred embodiment, the compound (a) is LAS191954, para-toluenesulfonate monohydrate and the immunobullous skin disease mediated by autoantibodies treated is pemphigus vulgaris.

Typically, the combinations and compositions as defined herein are for administration to a human or animal patient, preferably a human, canine, feline or equine patient, more preferably a human patient.

As discussed above, treatment of immunobullous skin diseases mediated by autoantibodies with phosphoinositide 3-Kinase delta (PI3K delta) inhibitors advantageously targets B-lymphocyte functions and reduces pathogenic IgG antibody titers against autoantigens associated with such diseases, specifically reducing the production of antibodies to Dsg3, which are associated with immunobullous skin diseases.

Thus, typically, the combinations and compositions as defined herein are for use in the treatment of an immunobullous skin disease mediated by autoantibodies as defined herein by one or more of:
prevention of B lymphocyte formation; and/or
attenuation of B cell function; and/or
reduction of the production of antibodies, typically antibodies to Dsg, preferably antibodies to Dsg3; and/or
reduction in the titer of autoantibodies, typically antibodies to Dsg, preferably antibodies to Dsg3.

The present invention therefore also provides a combination or composition as defined herein for use in prevention of B lymphocyte formation in a mammal, typically a human, suffering from an immunobullous skin disease mediated by autoantibodies as defined herein.

The present invention also provides a combination or composition as defined herein for use in attenuation of B cell function in a mammal, typically a human, suffering from an immunobullous skin disease mediated by autoantibodies as defined herein.

The present invention also provides a combination or compositions as defined herein for use in reduction of the production of antibodies, typically antibodies to Dsg in a mammal, typically a human, suffering from an immunobullous skin disease mediated by autoantibodies as defined herein. Preferably, the present invention provides a combination or composition as defined herein for use in reduction of the production of antibodies to Dsg3 in a mammal, typically a human, suffering from an immunobullous skin disease mediated by autoantibodies as defined herein.

The present invention also provides a combination or composition as defined herein for use in reduction in the titer of autoantibodies, typically antibodies to Dsg in a mammal, typically a human, suffering from an immunobullous skin disease mediated by autoantibodies. Preferably, the present invention provides a combination or composition as defined herein for use in reduction in the titer of antibodies to Dsg3 in a mammal, typically a human, suffering from an immunobullous skin disease mediated by autoantibodies as defined herein.

The pharmaceutical composition of the invention typically comprises a pharmaceutically acceptable carrier as defined herein.

In an embodiment of the present invention the pharmaceutical composition further comprises a therapeutically effective amount of one or more other therapeutic agents, as defined above.

Typically, each of the agents (a) and (b) in the compositions and combinations of the present invention, and the other optional therapeutic agents, may be administered together in the same pharmaceutical composition or in different compositions intended for separate, simultaneous, concomitant or sequential administration by the same or a different route. Typically, the pharmaceutical composition comprising the compound which is a phosphoinositide 3-kinase delta inhibitor is formulated for oral administration.

The pharmaceutical formulations may conveniently be presented in unit dosage form and may be prepared by any of the methods well known in the art of pharmacy.

The active compounds in the combinations of the invention may be administered by any suitable route, depending on the nature of the disorder to be treated, e.g. orally (as syrups, tablets, capsules, lozenges, controlled-release preparations, fast-dissolving preparations, etc); topically (as creams, ointments, lotions, nasal sprays or aerosols, etc); by injection (subcutaneous, intradermic, intramuscular, intravenous, etc.) or by inhalation (as a dry powder, a solution, a dispersion, etc).

Typically, the active compounds in the combinations of the invention are administered by a route other than topical administration.

Preferably, the active compounds in the combinations of the invention are administered orally.

Pharmaceutical compositions suitable for the delivery of compounds of the invention and methods for their preparation will be readily apparent to those skilled in the art. Such compositions and methods for their preparation can be found, for example, in Remington: The Science and Practice of Pharmacy, 21st Edition, Lippincott Williams & Wilkins, Philadelphia, Pa., 2001.

The pharmaceutically acceptable excipients which are admixed with the active compound or salts of such compound, to form the compositions of this invention are well-known per se and the actual excipients used depend inter alia on the intended method of administering the compositions. Examples, without limitation, of excipients include calcium carbonate, calcium phosphate, various sugars and types of starch, cellulose derivatives, gelatin, vegetable oils and polyethylene glycols.

Additional suitable carriers for formulations of the compounds of the present invention can be found in Remington: The Science and Practice of Pharmacy, 21st Edition, Lippincott Williams & Wilkins, Philadelphia, Pa., 2001.

Formulations of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A syrup formulation will generally consist of a suspension or solution of the compound or salt in a liquid carrier for example, ethanol, peanut oil, olive oil, glycerine or water with flavouring or colouring agent.

Where the composition is in the form of a tablet, any pharmaceutical carrier routinely used for preparing solid formulations may be used. Examples of such carriers include acacia, lactose, D-glucose (dextrose), sucrose, fructose, galactose, gelatine, starch, calcium carbonate, dibasic calcium phosphate, calcium sulphate, magnesium stearate, magnesium carbonate, isomalt, mannitol, maltitol, stearic acid, sorbitol, talc, xylitol, and mixtures thereof.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Where the composition is in the form of a capsule, any routine encapsulation is suitable, for example using the aforementioned carriers in a hard gelatine capsule. Where the composition is in the form of a soft gelatine capsule any pharmaceutical carrier routinely used for preparing dispersions or suspensions may be considered, for example aqueous gums, celluloses, silicates or oils, and are incorporated in a soft gelatine capsule.

Dry powder compositions for topical delivery to the lung by inhalation may, for example, be presented in capsules and cartridges of for example gelatine or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 2 μg and 150 μg of each therapeutically active ingredient. Alternatively, the active ingredient (s) may be presented without excipients.

Typical compositions for nasal delivery include those mentioned above for inhalation and further include non-pressurized compositions in the form of a solution or suspension in an inert vehicle such as water optionally in combination with conventional excipients such as buffers, anti-microbials, tonicity modifying agents and viscosity modifying agents which may be administered by nasal pump.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

The amount of each active which is required to achieve a therapeutic effect will, of course, vary with the particular active, the route of administration, the subject under treatment, and the particular disorder or disease being treated.

Effective doses are normally in the range of 0.01-2000 mg of active ingredient per day. Daily dosage may be administered in one or more treatments, preferably from 1 to 4 treatments, per day. Preferably, the active ingredients are administered once or twice a day, more preferably once a day.

When combinations of actives are used, it is contemplated that all active agents would be administered at the same time, or very close in time. Alternatively, one or two actives could be taken in the morning and the other (s) later in the day. Or in another scenario, one or two actives could be taken twice daily and the other (s) once daily, either at the same time as one of the twice-a-day dosing occurred, or separately. Preferably at least two, and more preferably all, of the actives would be taken together at the same time.

Preferably, at least two, and more preferably all actives would be administered as an admixture.

### Composition Example 1

50,000 capsules, each containing 50 mg each of LAS191954 methanesulfonate and prednisolone (active ingredients), were prepared according to the following formulation:

| | | |
|---|---|---|
| Active ingredients | 5 | Kg |
| Lactose monohydrate | 10 | Kg |
| Colloidal silicon dioxide | 0.1 | Kg |
| Corn starch | 1 | Kg |
| Magnesium stearate | 0.2 | Kg |

### Procedure

The above ingredients were sieved through a 60 mesh sieve, and were loaded into a suitable mixer and filled into 50,000 gelatine capsules.

### Composition Example 2

50,000 tablets, each containing 25 mg each of LAS191954 methanesulfonate and prednisolone (active ingredients), were prepared from the following formulation:

| | | |
|---|---|---|
| Active ingredients | 2.5 | Kg |
| Microcrystalline cellulose | 1.95 | Kg |
| Spray dried lactose | 9.95 | Kg |
| Carboxymethyl starch | 0.4 | Kg |
| Sodium stearyl fumarate | 0.1 | Kg |
| Colloidal silicon dioxide | 0.1 | Kg |

### Procedure

All the powders were passed through a screen with an aperture of 0.6 mm, then mixed in a suitable mixer for 20 minutes and compressed into 300 mg tablets using a 9 mm disc and flat bevelled punches. The disintegration time of the tablets was about 3 minutes.

Modifications, which do not affect, alter, change or modify the essential aspects of the compounds, combinations or pharmaceutical compositions described, are included within the scope of the present invention.

The following examples illustrate the invention

### Example 1—In Vitro Pharmacology Studies

The pharmacology of LAS191954 has been investigated in a range of in vitro studies.

### PI3Kδ Enzyme Residence Time

LAS191954 showed a residence time (time interval in which dissociation of 50% of the inhibitor occurs) in p1105 of 12 min or 17 min, whereas residence time was <1.4 min for the other three class I isoforms.

### Enzymatic and Cellular Potencies

Enzymatic potency on the four Class I PI3K recombinant human isoforms was determined by homogenous time-resolved fluorescence with a compound pre-incubation time of 30 min (Table 1). LAS191954 showed a potency on the target of 2.6 nM, with the highest selectivity versus PI3K p110α and the lowest versus PI3K p110γ and p110β, similarly.

TABLE 1
Enzymatic potencies of LAS191954 in the four PI3K isoforms

| Enzyme | IC₅₀ (nM) | Selectivity vs PI3Kδ (fold) |
|---|---|---|
| PI3K p110δ | 2.57 | 1 |
| PI3K p110α | 8220 | 3198 |
| PI3K p110β | 94.2 | 37 |
| PI3K p110γ | 71.7 | 28 |

Cellular potencies were determined in established cellular assays (Table 2). A primary PI3K5-dependent cellular assay was set up based on M-CSF-induced AKT phosphorylation, a downstream effector of PI3K5, in THP-1 cells. An IC₅₀ of 7.8 nM was obtained indicating that the compound was highly permeable. To evaluate the cellular inhibition of PI3K6, an assay based on stimulation of HUVEC cells with sphingosine-1-P was employed. The results indicated that the cellular selectivity for the δ isoform was 38-fold.

The main receptor on the surface of B cells is the BCR composed of a membrane immunoglobulin (Ig) and an Igα/Igβ heterodimer. The BCR is responsible for antigen recognition and binding. Signaling pathways associated with the BCR are crucial for B cell development, activation, proliferation, differentiation (e.g., memory and plasma B cells) and apoptosis. In naïve B cells, ligation of the BCR by cognate antigens initiates a series of responses/signal cascades that will induce cells to proliferate and differentiate, and will ultimately lead to the production of antibodies specific for the antigen. The PI3Kδ kinase is involved in the activation of B cells upon antigen binding to the BCR and thus inhibitors of PI3Kδ are expected to inhibit BCR activation in vitro.

The effect of LAS191954 on the function of human B cells was assessed in vitro by crosslinking the B-cell receptor with either anti-IgM or anti-IgD antibodies and assessing the early activation marker CD69 in the CD19+ B cell subset by flow cytometry. In isolated PBMC, the compound showed an IC₅₀ of 4.6 nM. Similar assays performed in a human whole blood context showed IC₅₀ of 47 nM for IgD and 34 nM for IgM. Plasma protein binding is the major factor accounting for the difference in potency between isolated PBMC and whole blood assays. These data indicate that LAS191954 is active in PBMCs in whole blood to inhibit B cell activation and antibody production.

In a functional assay on human neutrophils assessing the immune complex-induced ROS (reactive oxygen species) release, LAS191954 showed a potency of 11 nM, suggesting that PI3Kδ might be the only isoform involved in this effect.

TABLE 2
Cellular potencies of LAS191954

| | | | Isoform | IC₅₀ | Nbr |
|---|---|---|---|---|---|
| Cell type | Stimulus | Read out | involved | (nM) | tests |
| THP-1 | M-CSF | Phosphorylated | PI3Kδ | 7.8 | 2 |
| | | AKT | | | |
| HUVEC | S1P | Phosphorylated | PI3Kβ | 295 | 3 |
| | | AKT | | | |
| CD19+ cells | Anti-IgD | CD69 surface | PI3Kδ | 4.6 | 2 |
| (PBMC) | | expression | | | |
| CD19+ cells | Anti-IgD | B cell CD69 | PI3Kδ | 47 | 3 |
| (Human | | surface | | | |
| whole blood) | | expression | | | |
| CD19+ cells | Anti-IgM | B cell CD69 | PI3Kδ | 34 | 8 |
| (Human | | surface | | | |
| whole blood) | | expression | | | |
| Human | Immune | ROS release | PI3Kδ | 11 | 6 |
| neutrophils | complexes | | | | |

### General Selectivity

Activity of LAS191954 was assessed at a single concentration of 10 μM in:
81 GPCR receptors, 8 ion channels and 5 transporters (Cerep)
273 protein and lipid kinases (Millipore, Invitrogen and ProQinase)

No inhibition was found at the concentration tested.

### Cytotoxicity

In an assay assessing cytotoxicity of CHO cells after 24 h compound incubation, LAS191954 caused negligible cytotoxicity at all concentrations tested causing a maximum of 27% cell death at the highest tested concentration of 100 μM. This result indicates that the compound is not expected to be cytotoxic at estimated therapeutic plasma/tissue concentrations attained. No dose-response is observed across concentrations.

### Example 2—In Vivo Pharmacology Studies

The pharmacology of LAS191954 has been investigated in vivo using a range of studies listed in Table 3 below. The results of these studies are summarized in Table 4.

TABLE 3
In Vivo Studies of LAS191954

Effect Of Oral Administration Of LAS191954 on Concanavalin A
Induced Plasma Interleukin-2 (IL-2) Release In Wistar Rats
Evaluation of the Effect of LAS191954 On Primary and Secondary
T cell Dependent Antibody Response (TDAR) in Mice
Effect Of LAS191954 on A Murine Model Of Spontaneous
autoimmune disease

TABLE 4
Summary of In Vivo Studies Performed and
Inhibitory Doses Reported for LAS191954

| | | Type of | ID₅₀ |
|---|---|---|---|
| Study | Administration | model | (mg/kg) |
| Concanavalin A-induced IL2 | Single | Mechanistic | 0.13 |
| production in rats | | | |
| Primary IgM antibody | Repeated | Immune | 0.12 |
| responses to KLH | | response | |
| Primary IgG antibody | Repeated | Immune | 0.17 |
| responses to KLH | | response | |
| Secondary IgG antibody | Repeated | Immune | <0.3 |
| responses to KLH | | response | |
| Anti-Dsg3 and anti-dsDNA | Repeated | Autoimmune | <3 |
| autoantibody production in | | response | |
| Mrl/Lpr model | | | |

### LAS191954 Inhibits T-Cell Dependent Antibody Responses in Mice

The TDAR (T-Dependent Antibody Response) assay in mice using the KLH as antigen was selected to further explore the effect of LAS191954 on the function of the immune system. This assay allows a global assessment of the effect of a drug candidate on antigen presentation, helper T lymphocyte function and B lymphocyte dependent antibody production.

According to the kinetics of the specific antibody responses, the effect on primary specific IgM anti-KLH was analyzed on day +5 post immunization (PI) after 4 days of daily treatment with LAS191954 (0.03-10 mg/kg), and the effect on primary specific IgG was assessed on day +15 PI after 14-day dosing period (0.03-1 mg/kg). In both cases, the administration of the test compound started on the day of sensitization (day +1, KLH 2 mg/mouse, intravenously). LAS191954 induced a significant dose-dependent decrease in the primary IgM (ID₅₀=0.12 mg/kg) and IgG (ID₅₀=0.17 mg/kg) responses to KLH without apparent effects on the general health status of the animals. The decrease in the primary IgM anti-KLH response was accompanied by lower WBC counts mainly due to reduced number of peripheral blood lymphocytes. In contrast, no apparent effect on lymphocyte count was observed after treatment with LAS191954 in the study where specific IgG were analyzed. A possible reason for this discrepancy is that the lymphocyte count of the concurrent vehicle group of the latter study was abnormally lower than usual, which could mask a potential effect of the test compound on this parameter.

The effect of LAS191954 was subsequently assessed on the secondary TDAR assay in mice. This assay included two immunizations with KLH separated 15 days apart (50 μg KLH/animal, intraperitoneally) and specific IgG anti-KLH levels were measured on day +11 after the second immunization. Administration of test compound (0.3 and 3 mg/kg) started on the day of second immunization (day +1) and then once daily for the next 9 days. LAS191954 induced a significant decrease in secondary specific IgG anti-KLH response accompanied by reduced lymphocyte counts with an ID₅₀<0.3 mg/kg.

In the same TDAR assay protocols, a representative corticosteroid did not induce significant changes in the anti-KLH antibody response, while decreasing peripheral lymphocyte count and thymus weight.

### Example 3—Combination Studies of PI3Kδ Inhibitors and Corticosteroids

To assess the effect of PI3Kδ inhibition and corticosteroids (CS) in combination, several studies were performed and are summarized below in Table 5.

These models are examples of inflammation models in which combination effects and interactions between the mechanisms can be studied. The results obtained suggest that PI3Kδ inhibition represents a way to reduce CS insensitivity in conditions that classically require high dose CS for treatment, such as pemphigus.

TABLE 5
List of studies performed with LAS191954 in combination with CS

| | | Doses in | | |
|---|---|---|---|---|
| | | combination | | |
| Model | Species | tested | Read out | Results |
| ConA- | Rat | LAS191954 | IL2 | LAS191954 |
| induced | | 0.1 mg/kg + | production | only = 49% |
| IL2 | | Dexamethasone | | reduction in IL2 |
| | | 0.03 mg/kg | | Dexamethasone |
| | | | | only = 42% |
| | | | | reduction in IL2 |
| | | | | LAS191954 + |
| | | | | Dexamethasone = |
| | | | | 80% reduction |
| | | | | in IL2 |
| House | Mouse | LAS191954 | Neutrophils, | LAS191954 |
| dust mite | | 0.1-10 mg/kg + | eosinophils | only = Modest |
| induced | | Dexamethasone | and | dose-dependent |
| asthma | | 1 mg/kg | lymphocytes | inhibition of |
| model | | Dexamethasone | in broncho- | eosinophils and |
| | | 0.1-10 mg/kg + | alveolar | lymphocytes, but |
| | | LAS191954 | fluid (BALF) | increase in |
| | | 1 mg/kg | | neutrophils |
| | | | | Dexamethasone |
| | | | | only = 53%, |
| | | | | 58%, and 64% |
| | | | | reduction in |
| | | | | neutrophils, |
| | | | | eosinophils and |
| | | | | lymphocytes |
| | | | | LAS191954 + |
| | | | | Dexamethasone = |
| | | | | 72%, 93%, and |
| | | | | 98% reduction |
| | | | | in neutrophils, |
| | | | | eosinophils and |
| | | | | lymphocytes |
| Cigarette | Mouse | LAS191954 | Neutrophils | LAS191954 |
| smoke | | 1 mg/kg + | in BALF | only = 49% |
| model | | Dexamethasone | | reduction in |
| | | 0.3 mg/kg | | neutrophils |
| | | | | Dexamethasone |
| | | | | only = 11% |
| | | | | reduction in |
| | | | | neutrophils |
| | | | | LAS191954 + |
| | | | | Dexamethasone = |
| | | | | 73% reduction in |
| | | | | neutrophils |

A combination study of LAS191954 and a corticosteroid (dexamethasone) was performed in a rat model of con A induced IL2 production in which the compound was administered one hour prior to intravenous con A challenge and IL2 measured 90 minutes later. For this purpose, rats were administered LAS191954 at 0.1 mg/kg and/or dexamethasone at 0.03 mg/kg using the same protocol as described above. These doses were selected as the ones providing around a 50% inhibition for both mechanisms. Analysis of plasma levels confirmed that both compounds attained the same levels when administered alone or in combination, suggesting no pharmacokinetic interaction. LAS191954 and dexamethasone caused a 49% and a 42% inhibition of IL2 production, respectively, versus vehicle-treated con A-induced rats. In contrast, concomitant administration of both compounds caused an 80% inhibition of IL2 production, suggesting that both mechanisms act independently and produce additive effects.

### Example 4—Inhibition of Specific Dsg3 Autoantibody Production in a Spontaneous Autoimmune Disease Model

The MRL/Ipr mouse model was selected as a model of efficacy to demonstrate amelioration of autoimmune-related features, in particular, production of autoantibodies. The primary endpoint of this study was assessment of autoantibody production, including pemphigus-specific anti-Dsg3 antibodies.

Mice were randomized to receive vehicle alone, 3 mg/kg LAS191954, or 10 mg/kg prednisolone orally once a day for 6 weeks. The dose of LAS191954 was selected to ensure complete PI3Kδ coverage for 24 h when administered once a day. The prednisolone dose was selected based on previous reports and corresponds to a high CS dose in humans.

As autoantibodies develop progressively and may follow a different course in each animal, anti-dsDNA antibody levels were measured on week 12 and used to uniformly distribute animals to dosing groups. At week 13, daily treatments were initiated and continued for 6 weeks. Antibodies to dsDNA and Dsg3 were measured at weeks 12, 15, 17 and 19. Skin lesions were inspected visually throughout the study. Effects on other parameters such as proteinuria, as well as general hematological, serological, and histological signs were evaluated at study completion.

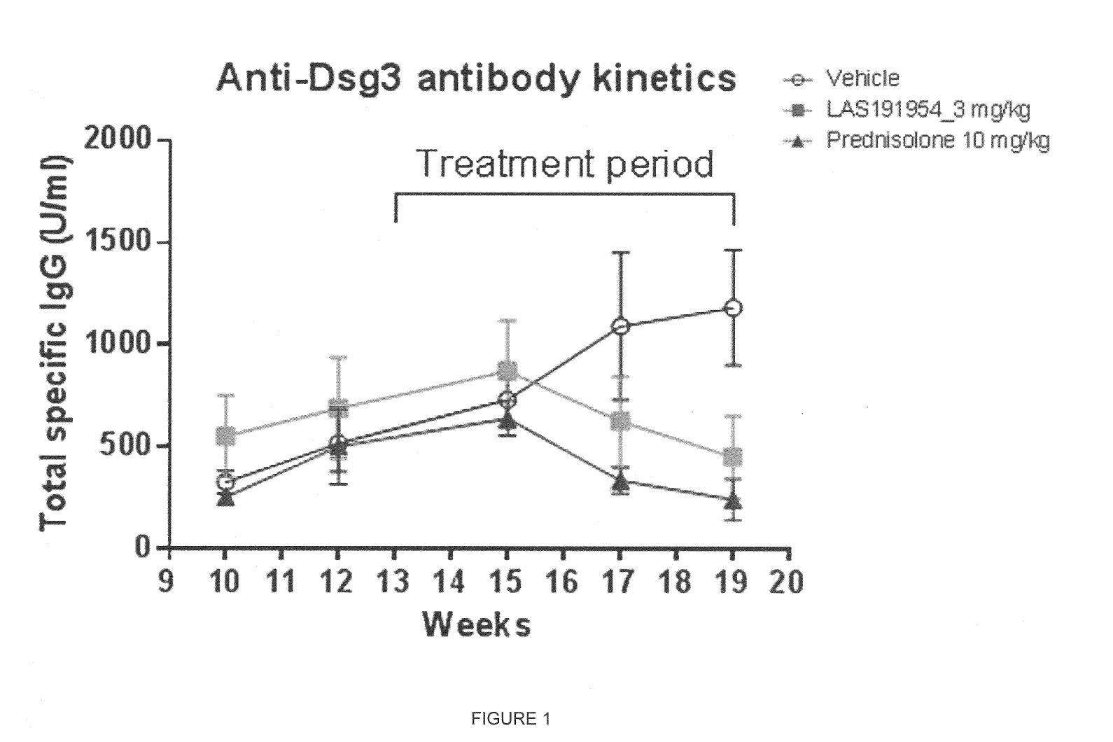

Kinetic analysis of autoantibody production demonstrated that anti-dsDNA antibody levels were approximately 2,000-fold higher than anti-Dsg3 antibodies and increased steadily between weeks 12 and 19. Daily administration of LAS191954 for 6 weeks at a dose of 3 mg/kg significantly reduced anti-dsDNA and anti-Dsg3 antibody production (see FIGS. 1&2). When the Area Under Curve encompassing weeks 12 to 19 was calculated and normalized for initial week 12 antibody titer for each individual, LAS191954 led to a 47.5% and a 66% inhibition of anti-Dsg3 and anti-dsDNA antibodies, respectively, similar to prednisolone (49.5 and 47%, respectively) (Table 6).

TABLE 6
Inhibition (%) of Autoantibody Production
(Normalized AUCw 12-19) versus Vehicle

| | LAS191954 3 mg/kg | Prednisolone 10 mg/kg |
|---|---|---|
| Anti Dsg3 total IgG | 47.5 ± 10.4% * | 49.5 ± 10% * |
| Anti dsDNA total IgG | 65.6 ± 3.1% *** | 46.9 ± 10.9% ** |

*** p < 0.001;
** p < 0.01;
* p < 0.05 using one-way ANOVA and Dunnet post-test versus vehicle group

When absolute specific IgG levels were measured, LAS191954 reduced the average levels of anti-dsDNA and anti-Dsg3 specific IgGs on the last week of administration below those at the start of treatment (Table 7).

TABLE 7
Absolute Specific IgG Levels at the Beginning and End of Treatment
Absolute IgG levels (U/ml) (Mean ± SEM)

| | Anti-dsDNA | | Anti-Dsg3 | |
|---|---|---|---|---|
| | Week 12 | Week 19 | Week 12 | Week 19 |
| Vehicle | 369.253 ± 52.919 | 2,750.673 ± 745.899 | 515 ± 140 | 1185 ± 284 |
| LAS191954 | 370.152 ± 52.401 | 333.806 ± 106.959 | 687 ± 252 | 450 ± 201 |
| 3 mg/kg | | | | |
| Prednisolone | 372.796 ± 56.638 | 933.755 ± 343.424 | 501 ± 188 | 242 ± 101 |
| 10 mg/kg | | | | |

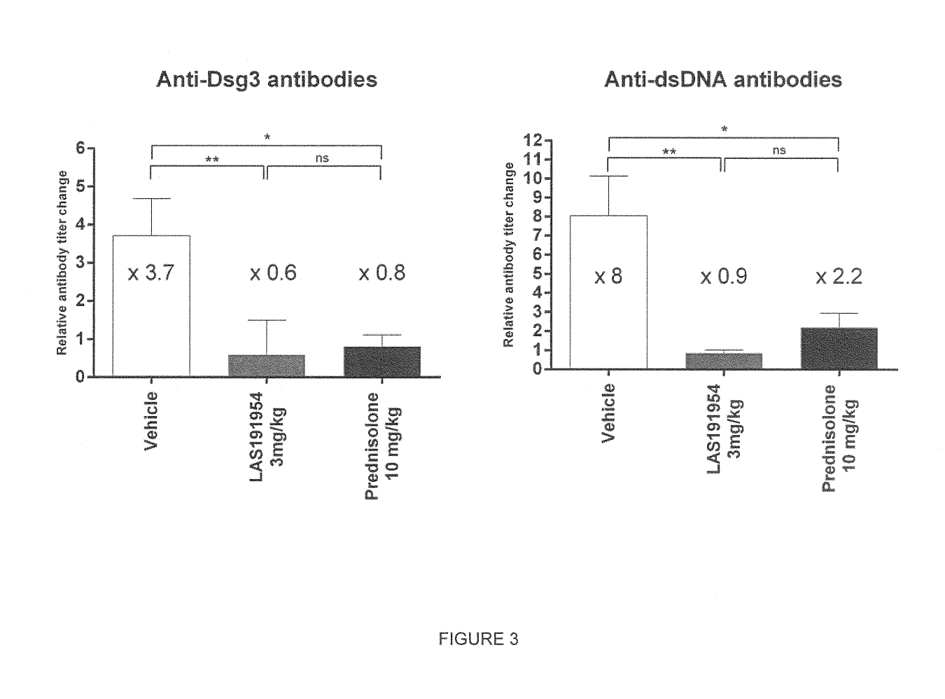

FIG. 3 shows the fold change in antibody titers at week 19 versus titers at the initiation of treatment. Whereas the anti-Dsg3 antibody titers increased approximately four-fold in the vehicle treated animals, LAS191954 and prednisolone induced a mean decrease of 40% and 20%, respectively, in antibody levels below those at the beginning of treatment. Likewise, anti-dsDNA antibody titers increased about 8-fold, whereas LAS191954 caused a 10% reduction and prednisolone doubled the levels at the end of treatment. For each individual, the ratio between antibody titer at Week 19 and that at Week 12 was calculated. (Values represent mean of ratios for each treatment group±SEM. *p<0.05; **p<0.01; ns nonstatistically significant.)

This demonstrates that prolonged daily treatment with LAS191954 is able to significantly reduce anti-Dsg3 autoantibody production in a spontaneous model of autoimmune disease that does not rely on active immunization. Antibodies to both dsDNA and most importantly Dsg3, the specific antigen in PV, were reduced with similar efficiency.

### Example 5—Immunization-Induced Mouse Model of Epidermolysis Bullosa Acquista (EBA)

LAS191954 was tested in an immunization-induced mouse model of epidermolysis bullosa acquista (EBA) in B6.SJL-H2s mice to demonstrate the link between PI3Kδ inhibition and amelioration of autoantibody-mediated cutaneous lesions.

### Materials and Methods

### Animal Experiments

Induction of experimental EBA was performed as described in Iwata H, Bieber K, Tiburzy B et al., J Immunol. 2013; 191:2978-2988. Briefly, 6-10 week B6.SJL-H2s mice were immunized with an emulsion of a recombinant protein encompassing the vWFA2 binding domain of mouse type VII collagen (COL7) in adjuvant (Titermax). After immunization, mice were weekly evaluated for the presence and extend of clinical disease, measured as percentage of body surface affected by skin lesions (erythema, blisters, erosions and crusts). When 2% or more of the body surface area was affected by skin lesions, the mouse was randomly allocated to one of the treatment groups:
Vehicle to serve as untreated control (n=5)
Methylprednisolone (MP, orally at 20 mg/kg/day) to serve as reference treatment (n=6)
LAS191954 orally at 3 mg/kg/day (n=6)

Treatments were carried out over a 6-week period, and mice were evaluated for the extend of clinical disease (primary endpoint) weekly. Clinical manifestations were scored 0 to 5, corresponding to 0%, <1%, ≧1% to <5%, ≧5% to <10%, ≧10% to <20% of body surface area affected, respectively. Area under Curve (AUC) was calculated from the score at inclusion, 1, 2, 3, 4, 5 and 6 weeks after allocation to treatment. For better comparability between experiments, the affected body surface area at weeks 1-6 was related to that at inclusion (set at 1).

Body weight was monitored weekly during treatment.

### Results

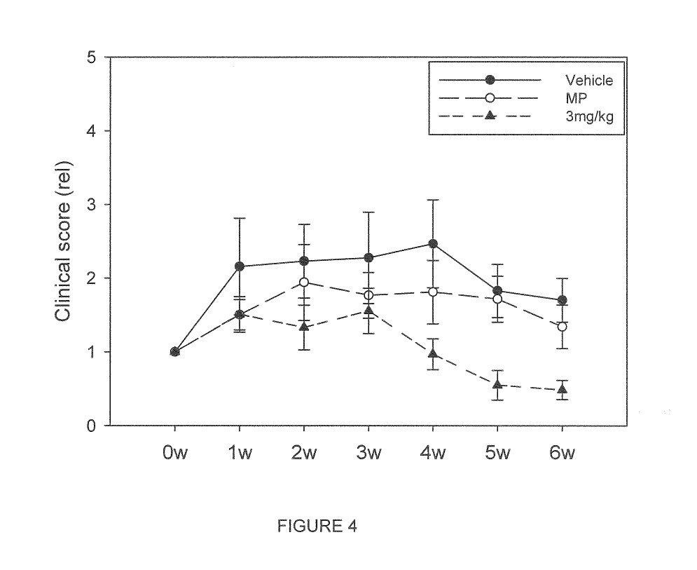

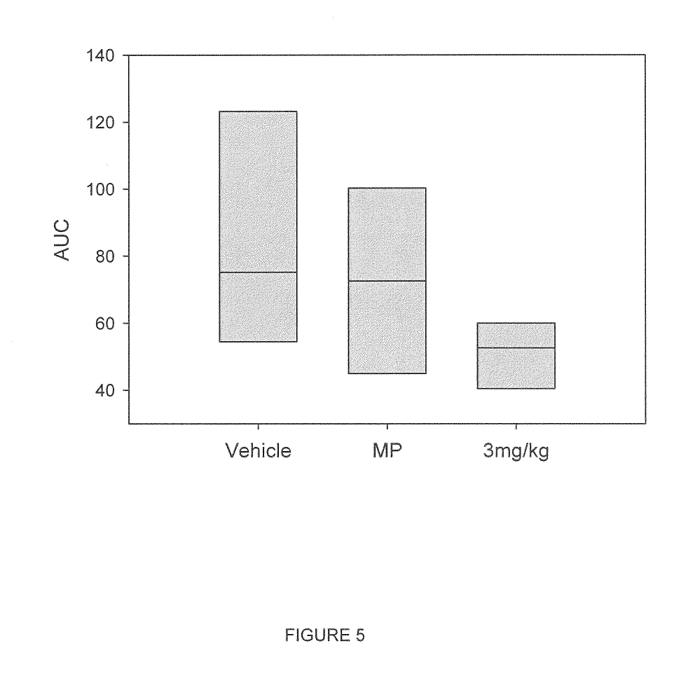

In vehicle-treated mice, the relative clinical score increased from 1 to 1.7 at the end of the 6 week treatment period with a maximum index of 2.5 observed at 4 weeks of treatment (FIGS. 4 and 5).

FIG. 4 shows the percentage of body surface area affected by skin lesions in relation to the score at inclusion to treatment. Disease severity increases in vehicle-treated group during the 6 week treatment period. Methylprednisolone modestly reduced clinical severity during the 6 week treatment period versus the vehicle-treated group, although it was not statistically significant. In contrast, LAS191954 progressively and significantly (p<0.001 for weeks 4, 5 and 6) reduced the clinical severity over the same period, obtaining a final score below the initial one, i.e. even beyond the initial clinical score (mean±SEM), indicating a clear trend towards normalization.

FIG. 5 shows the overall disease activity, expressed as AUC derived from graphs in FIG. 4. (Median±quartiles). In accordance with the time-course results, Area Under Curve calculation showed a significant reduction in the accumulative clinical score over time with LAS191954 treatment versus vehicle.

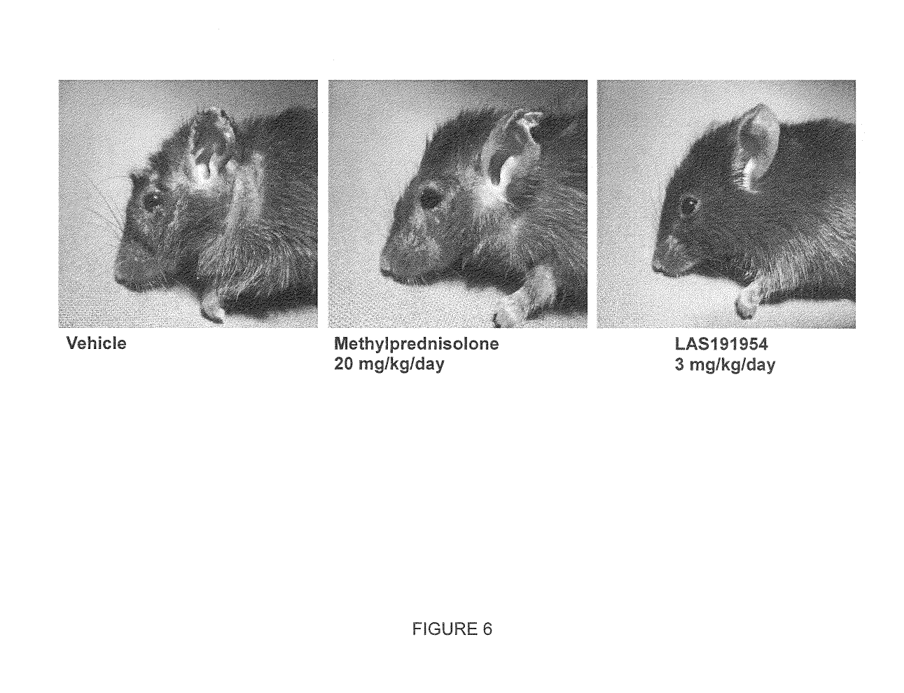

FIG. 6 shows representative clinical manifestations of the three treatment groups at the end of the treatment period.

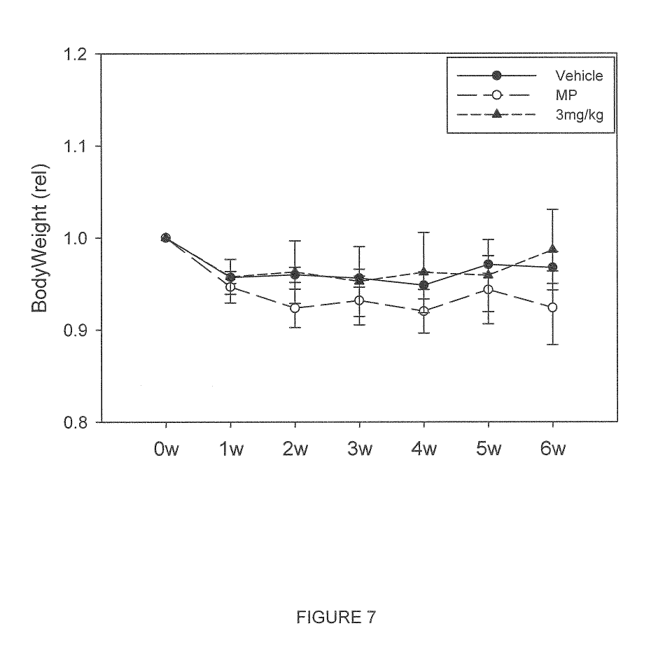

Body weight gain was not altered by LAS191954 administration over time. In contrast, methylprednisolone diminished the body weight gain especially at the beginning of treatment (FIG. 7). The LAS191954-treated group showed a similar behavior to the vehicle-treated group with modest gain weights along the treatment period. The methylprednisolone-treated group showed lower gain weight than the vehicle group, especially during the first two weeks of treatment.

### CONCLUSION

LAS191954 ameliorates the cutaneous disease manifestations in an induced model of epidermolysis bullosa acquisita, an autoantibody-mediated bullous disease model. The effect is better than that induced by treatment with a high dose corticosteroid and shows a clear trend towards time-dependent clinical normalization. Taken together, these results provide a direct link between PI3Kδ inhibition and clinical efficacy in a cutaneous bullous disease.

### Example 6—Effect of Combining PI3Kδ Inhibitors and Corticosteroids

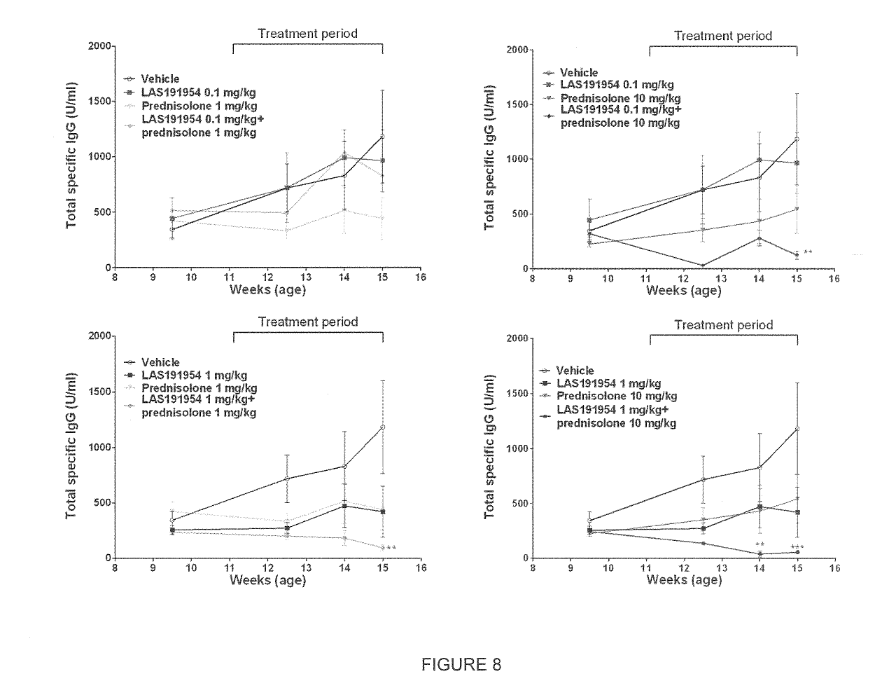

The effect of combining PI3Kδ inhibitors and corticosteroids was tested in Mrl/Lpr mice (see FIG. 8)

Groups of originally 9.5-week age female MRL/Ipr mice were treated once a day for 28 days with LAS191954 (1 and 0.1 mg/kg), prednisolone (10 and 1 mg/kg) or combinations of both. The kinetics of anti-Dsg3 auto-antibody production was followed during treatment. Combinations of prednisolone and LAS191954 showed a clear additive effect versus each compound alone on the anti-Dsg3 antibody titer over the course of the study. Specifically, three out of the four combinations tested increased the maximal efficacy observed with prednisolone. The combination of the lower dose of LAS191954 (0.1 mg/kg) and lower dose of prednisolone (1 mg/kg) did not show a significant difference on the production of autoantibodies versus both doses in monotherapy. However, the same dose of LAS191954 when combined with the highest dose of prednisolone significantly decreased the anti-Dsg3 antibody titers. This observation is corroborated in two other combinations, where the addition of an efficacious dose of LAS191954 to both prednisolone doses provided higher efficacy than each compound alone.

1. A pharmaceutical composition comprising (a) a compound chosen from an inhibitor of phosphoinositide 3-kinase delta or a pharmaceutically acceptable salt and/or solvate thereof, and (b) a corticosteroid.

2. The composition according to claim 1, wherein the compound is chosen from LAS191954, idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, XL-499, KAR-4141, RP-5090, PWT-143, IPI-443, RP-6503, ONO-146040, SPR-965, LOR-220, SF-2626, X-339, X-480, PQR-401, INCB-050465, LS-008, CLR-457, PCN-5603, 7-hydroxystaurosporine, PF-04691502, TG-100115, BGT-226, SF-1126, PKI-179, panulisib, or a pharmaceutically acceptable salt and/or solvate thereof.

3. The composition according to claim 1, wherein the corticosteroid is chosen from: prednisolone, methylprednisolone, dexamethasone, dexamethasone cipecilate, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone furoate, rimexolone, prednisolone farnesylate, ciclesonide, butixocort propionate, deprodone propionate, fluticasone propionate, fluticasone furoate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate and hydrocortisone probutate.

4. The composition according to claim 1 further comprising at least one additional active compound chosen from:
a) Dihydrofolate reductase inhibitors,
b) Immunosuppressants,
c) Anti-tumor necrosis factor-alpha (Anti-TNF-alpha) monoclonal antibodies,
d) Soluble Tumor necrosis factor-alpha (TNF-alpha) Antagonists,
e) Anti-CD20 (lymphocyte protein) monoclonal antibodies,
f) Anti-BAFF/BlyS,
g) Anti-TACl,
h) Anti-BAFF receptor,
i) Anti-CD19,
j) Anti-ICOSL,
k) Anti-FasL monoclonal antibodies,
l) Btk inhibitors,
m) Calcineurin inhibitors,
n) Inosine-monophosphate dehydrogenase (IMPDH) inhibitors,
o) Tetracyclines, and
p) Dihydropteroate synthase inhibitors.

5. (canceled)

6. A method for treating a subject afflicted with a skin disease comprising administering to said subject an effective amount of the composition according to claim 1.

7. The method according to claim 6, wherein the skin disease is an immunobullous skin disease mediated by autoantibodies.

8. The method according to claim 6, wherein the skin disease is chosen from pemphigus vulgaris, pemphigus vegetans, pemphigus foliaceus, endemic pemphigus foliaceus, intercellular IgA dermatosis, paraneoplastic pemphigus, bullous pemphigoid, mucous membrane pemphigoid, pemphigoid gestationis, linear IgA disease, epidermolysis bullosa acquisita, bullous systemic lupus erythematosus and dermatitis herpetiformis.

9. A product comprising a pharmaceutical composition according to claim 1, optionally together with at least one additional active compound chosen from:
a) Dihydrofolate reductase inhibitors,
b) Immunosuppressants,
c) Anti-tumor necrosis factor-alpha (Anti-TNF-alpha) monoclonal, antibodies,
d) Soluble Tumor necrosis factor-alpha (TNF-alpha) Antagonists,
e) Anti-CD20 (lymphocyte protein) monoclonal antibodies,
f) Anti-BAFF/BlyS,
g) Anti-TACl,
h) Anti-BAFF receptor,
i) Anti-CD19,
j) Anti-ICOSL,
k) Anti-FasL monoclonal antibodies
l) Btk inhibitors,
m) Calcineurin inhibitors,
n) Inosine-monophosphate dehydrogenase (IMPDH) inhibitors,
o) Tetracyclines, and
p) Dihydropteroate synthase inhibitors
for simultaneous, concurrent, separate or sequential use in the treatment of a human or animal body.

10. A method for treating a subject afflicted with a skin disease comprising administering to said subject an effective amount of the product according to claim 9, wherein the pharmaceutical composition is administered to the subject simultaneous, concurrent, separate or sequential with the at least one additional active compound.

11. A method for treating a subject afflicted with a skin disease comprising administering to said subject an effective amount of an inhibitor of phosphoinositide 3-kinase delta or a pharmaceutically acceptable salt and/or solvate thereof simultaneous, concurrent, separate or sequential with a corticosteroid and optionally simultaneous, concurrent, separate or sequential with at least one additional active compound chosen from:
a) Dihydrofolate reductase inhibitors,
b) Immunosuppressants,
c) Anti-tumor necrosis factor-alpha (Anti-TNF-alpha) monoclonal, antibodies,
d) Soluble Tumor necrosis factor-alpha (TNF-alpha) Antagonists,
e) Anti-CD20 (lymphocyte protein) monoclonal antibodies,
f) Anti-BAFF/BlyS,
g) Anti-TACl,
h) Anti-BAFF receptor,
i) Anti-CD19,
j) Anti-ICOSL,
k) Anti-FasL monoclonal antibodies
l) Btk inhibitors,
m) Calcineurin inhibitors,
n) Inosine-monophosphate dehydrogenase (IMPDH) inhibitors,
o) Tetracyclines, and
p) Dihydropteroate synthase inhibitors.

12. (canceled)

13. (canceled)

14. (canceled)

15. (canceled)
